(19) United States
(12) Patent Application Publication
(10) Pub. No.: US 2012/0329675 A1
(43) Pub. Date: Dec. 27, 2012

(54) Testing of Biofilm for Anti-microbial Agent Susceptibility

(76) Inventors: Merle E. Olson, Calgary (CA); Howard Ceri, Calgary (CA)

(21) Appl. No.: 13/384,683

(22) PCT Filed: Jul. 20, 2010
(86) PCT No.: PCT/CA2010/001151

§ 371 Date: Aug. 27, 2012

Related U.S. Application Data
(60) Provisional application No. 61/227,039, filed on Jul. 20, 2009.
(60) Provisional application No. 61/358,870, filed on Jun. 25, 2010.

(51) Int. Cl. C40B 30/06; C12Q 1/20

(52) U.S. Cl. 506/10; 435/33

(57) ABSTRACT

This invention is an apparatus and method for susceptibility testing one or more biofilms, for selecting one or more anti-microbial combinations with efficacy against the biofilm, and/or in treating a disease or condition mediated by the biofilm The invention includes methods for the selection of antibiotic combinations with efficacy against a specific microbial type and for the formulation of microbe-specific test plates. The invention also includes an assay system to test patient specific isolates for sensitivity to the anti-microbial combinations.

### FIELD OF THE INVENTION

This invention relates to improved methods and devices for the analysis of biofilms, and to determining microbial sensitivity or susceptibility to anti-microbial or anti-biofilm reagents, preferably combinations of anti-biofilm reagents, such as antibiotics or biocides. In a preferred embodiment of the invention, methods and devices include selecting appropriate individual and combinations of anti-biofilm agents with enhanced efficacy for determining susceptibility of one or more microorganisms to one or more anti-biofilm agents.

In accordance with the present invention, determining susceptibility provides clinical information and guidance appropriate for the treatment of biofilm-mediated disease, including but not limited to Pseudomonas aeruginosa, specifically lung infections in cystic fibrosis (CF) patients.

This invention provides methods and devices for the selection of appropriate anti-biofilm agents with enhanced efficacy for the treatment of CF. The invention also provides methods and devices for selecting an antibiotic or combination of antibiotics for the treatment of CF in a specific patient.

### BACKGROUND OF THE INVENTION

Standardized susceptibility testing, which is based on the minimum inhibitory concentration (MIC), has guided drug discovery and clinical antibiotic selection for decades. The crux of the MIC test is to identify the lowest concentration of an antimicrobial agent that is required to inhibit planktonic bacterial growth in a liquid culture¹ The standardized MIC assay—which is used worldwide—has a good track record of predicting treatment outcome for a variety of acute infections. However, there are certain circumstances in which the prognostic ability of these assays is limited, particularly with chronic infections hypothesized to have a biofilm etiology.

During biofilm formation, microbes aggregate with each other or may adhere to a surface, encasing themselves in a self-produced matrix of extracellular polymers. This occurs in a tightly regulated response to environmental cues² and results in physiological and genetic diversification of the cells in the biofilm³⁻⁶. This cellular diversity is linked to an increase in antimicrobial resistance and tolerance of the microbial population. Because of this, biofilms are thought to be responsible for many chronic or device-related infections that are recalcitrant topersonalized antibiotic therapy based on MIC testing^{6,9,10}. The simplest example of this is single-species biofilms formed by nontypeable Haemophillus influenzae in the inner ear of children with chronic otitis media with effusion (OME)¹¹. Antibiotic therapies for OME guided by standardized MIC testing generally show short-term therapeutic benefit, but little long-term efficacy¹². Antibiotic susceptibility testing of nontypeable H. influenzae biofilms predicts different antibiotic combinations than MIC testing for the treatment of OME¹³, and some of these drug regimens are currently being studied for treatment of this chronic disease. Thus, there is an increasing need for laboratory technologies to accurately assess the susceptibility of biofilms to antimicrobial agents during diagnostic testing.

In addition to this demand, biofilm susceptibility test methods are also required to develop biocides that can eliminate microbial biofilms from hard surfaces in a wide range of industrial and agricultural settings. Moreover, there is a growing demand for simple biofilm models in basic microbiological research. To address these needs, several in vitro biofilm models have been developed.

The most widespread systems used to grow biofilms in laboratories are flow cells, drip flow reactors, spinning-disk and tube biofilm reactors. These models have several advantages in common, including growth of biofilms to high population densities, high biomass yields, continuous culture conditions and controlled fluid dynamics. However, these systems are hampered by an inability to produce more than a few biofilm samples at one time. Moreover, as these reactors depend on continuous flow, they require large volumes of culture medium to operate and are somewhat prone to contamination or leakage. To enable small-volume, high-throughput experimental approaches, two batch culture methods have been developed, namely, (i) cultivation of biofilms directly in microtiter plates¹⁴⁻¹⁶ and (ii) growth of biofilms on peg lids¹⁷. A simple, yet versatile, apparatus for cultivating biofilms on peg lids is the Calgary Biofilm Device (CBD) (or minimum biofilm eradication concentration (MBEC) assay.

The characterization of microorganisms has traditionally employed methods of batch culture studies, where the organisms exist in a dispersed or planktonic state. Over the past 25 years, it has been recognized that the major component of the bacterial biomass in many environments are sessile bacteria, e.g., in biofilms, and that the growth of organisms in biofilms is physically and physiologically different than growth of the same organisms in batch culture. These differences contribute to observed alterations in both the pathogenesis of these organisms and their susceptibilities to antimicrobial agents. The antibiotic resistance is generally attributed to the production of a protective exopolysaccharide matrix and alterations in microbial physiology.

P. aeruginosa, which is a gram-negative rod, and its associated biofilm structure has far-reaching medical implications and is the basis of many pathological conditions. P. aeruginosa is an opportunistic bacterium that is associated with a wide variety of infections, e.g., chronically colonizes the lung of patients with cystic fibrosis. Pseudomonas aeruginosa growing as biofilms are highly resistant to antibiotics and are resistant to phagocytes.

The inventors have developed assays with a specific purpose of identifying anti-biofilm agents and anti-biofilm agent combinations that are effective in eliminating and controlling any gram-negative bacterium, including but not limited to E. coli, Burkholderia spp., Acinetabacter spp., Proteus spp, Salmonella spp. Stenotrophomonas spp, Vibrio spp, Yersinia spp, Campylobacteria spp., and Pseudomonas spp. biofilms. Such a product improves the selection of antimicrobial drug therapy for patients with a disease or condition mediated by a gram-negative bacterium.

It is now widely known that bacteria in the form of biofilms are more resistant to antibacterial reagents than planktonic bacteria. Yet testing for the presence of bacteria and the testing the efficacy of antibiotics against bacteria has traditionally involved testing for planktonic bacteria. Studies have shown a greater than hundred-fold resistance to antibiotics of biofilms when compared to the same bacteria in a planktonic (free floating) state. This resistance is multi-factorial due to many phenotypic adaptations as part of the biofilm mode of growth, including but not limited to the mucopolysaccharide coating that is developed, and a physiological alteration in the microorganism.

Selecting antibiotics and combinations of antibiotics for treating biofilm infections continues to rely on minimal inhibitory concentration (MIC) assays despite the recognized lack of efficacy of these tests. Some have suggested the use of biofilm inhibitory concentrations (BIC) (Moskowitz, et al.; J. Clin. Microbiology, 42:1915-1922 (May 2004)), but the evidence suggests that both BIC and MIC address planktonic bacteria, not sessile bacteria.

In contrast, the present invention uses sonication or re-growing biofilm on a separate recovery plate in its processing so that the complete, intact biofilm can be obtained and assayed. Also, the processes of the present invention include growing the biofilm under dynamic or flowing conditions, and neutralizing the anti-microbials, both of which individually and collectively fortify any assay results.

Therefore a need exists for improved processing and assaying devices and methods for selecting effective compositions against gram-negative bacteria, including anti-biofilm compositions that are effective against gram-negative biofilm mediated conditions and infections.

### SUMMARY OF THE INVENTION

The invention comprises improved methods and devices for the selection of one or more active agents, either alone or in combination, effective against biofilm formed by one or more gram-negative bacteria. In preferred embodiments of the invention, the devices and methods may be used in the treatment of a biofilm infection. In the most preferred embodiments of the invention, the methods and devices may be used in the diagnosis and treatment of cystic fibrosis.

The biofilm may be any gram-negative biofilm, including but not limited to those formed from E. coli, Burkholderia spp, Acinetobacter spp, Proteus spp, Salmonella spp. Stenotrophomonas spp and Pseudomonas spp, Vibrio spp, Yersinia spp, Campylobacteria spp.; other additional bacteria, fungi, or algae, viruses, and parasites; or a microorganism that is incorporated within a biofilm as it is formed; or mixed biofilms, e.g., containing more than one bacterial, viral, fungal, parasitic, or algal biofilm. In preferred embodiments of the invention, the Pseudomonas species is Pseudomonas aeruginosa. As shown by the examples, the methods and devices of the present invention are generic for any gram-negative bacterium species and biofilm, including combinations of gram-negative bacterium species and biofilms.

The devices and methods of the present invention also include developing a treatment protocol. In preferred embodiments, the treatment protocol can be tailored to a specific patient and or may form the basis of developing a personalized medical treatment or approach.

The devices and methods of the present invention are effective in treating any gram-negative species. The devices and methods are also effective in treating diseases and/or medical conditions caused or mediated by a gram-negative bacterium. The invention also provides a clinically significant assay tailored to growing a particular biofilm or biofilms, and to determining the appropriate active agent or agents effective against that biofilm. In preferred embodiments of the invention, the assay provides the minimum biofilm eradication concentration (MBEC), the minimum inhibitory concentration (MIC), or the minimum biocidal concentration (MBC), or combinations thereof In the most preferred embodiments of the invention, the susceptibility assay and devices provide MBEC, MBC, and MIC values in combination, that is in a single assay protocol.

The invention also provides an easy, economical, and clinically significant assay that can be conducted over a wide interval between tests, e.g., every six months, so that the clinician can determine if there is a change in the patient's condition that warrants a change in the treatment. In these embodiments of the invention, a biological specimen from a patient is tested using an assay device of the present invention, the appropriate treatment is determined, then, after a predetermined interval (e.g., several months), a biological specimen from the patient is tested using an assay device of the present invention, and any changes to the treatment protocol are determined.

The present invention provides a panel of individual and/or combined active agents for selecting a composition containing one or more active agents with efficacy against one or more gram-negative biofilms. These agents or combination of agents may be useful in treating patient-specific infectious organisms. The present invention provides a method and apparatus for the selection of combinatorial antibiotic treatment of biofilm associated infectious diseases. As used herein combinatorial refers to combining a first active agent with at least one second active agent. The active agents may be an antibiotic, a pharmaceutical, a biological, a chemical, or any other agent that provides a beneficial result in the treatment of a gram-negative bacterium and/or a disease or condition mediated by the gram-negative bacterium.

The devices and methods of the present invention may also be useful in determining and developing a pharmaceutical composition specific for anti-microbial therapeutic use on an individual patient. In preferred embodiments of the invention, the devices and methods are used to determine and develop a treatment protocol for a patient suffering from a disease or infection caused by a gram-negative biofilm, e.g., a Pseudomonas species and/or a patient suffering from CF. The devices and methods of the present invention also provide an alternative to existing treatments that contribute to well-publicized antibiotic resistance.

The devices and methods of the present invention may also be used to identify genetic shift, antibiotic resistance, and genetic variations in the process of developing the appropriate treatment protocol tailored for the particular patient. In these embodiments of the invention, the devices and methods of the present invention are used over a defined time interval, including but not limited to daily, every month, every two months, every six months, and/or annually. In these embodiments of the invention, the treatment protocol may be confirmed or changed according to the results of any subsequent assay.

The invention also provides an in vitro assay tailored to the presence of a biofilm, namely an assay based on determining the minimum biofilm eradication concentration (MBEC). In preferred embodiments of the invention, the devices and methods provide any combination of MBEC, minimum inhibitory concentration (MIC), and minimum biocidal concentration (MBC) values.

The devices and methods of the present invention are improved over prior art devices in one or more of the following: the device and process involve testing intact biofilm; using sonication to remove the intact biofilm; the devices and process apply to a wider range of gram-negative biofilms, the anti-biofilm agent covers a wider range of agents, including biocides, etc.; the devices and methods are high-throughput and therefore more efficient and cost effective; growing the biofilm is improved, involving increased understanding and application of process conditions to enhance biofilm growth; and the devices and methods may be adapted or configured to test the susceptibility of two or more bacteria on a single plate (or device assembly) and/or with one or more anti-biofilm agents.

The invention also includes the use of an integrated device or assembly, multiple or plural assemblies, multiple or plural sub-assemblies, or combinations thereof.

Batch culture of biofilms on peg lids is a versatile method that can be used for microtiter determinations of biofilm antimicrobial susceptibility. The present invention teaches this versatile method and a set of parameters (e.g., surface composition, the rate of rocking or orbital motion, temperature, cultivation time, inoculum size, atmospheric gases and nutritional medium) that can be adjusted to grow single- or multispecies biofilms on peg surfaces. Mature biofilms formed on peg lids can then be fitted into microtiter plates containing test agents. After a suitable exposure time, biofilm cells are disrupted into a recovery medium using sonication. Microbiocidal end points can be determined qualitatively using optical density measurements or quantitatively using viable cell counting. Once equipment is calibrated and growth conditions are at an optimum, the procedure typically involves about five hours of work over four to six days. This method allows antimicrobial agents and exposure conditions to be tested against biofilms on a high-throughput scale.

Originally described by Ceri et al. ¹⁷, growth of biofilms on peg follows a core protocol with several discrete parameters that can be adjusted to facilitate biofilm growth for a variety of bacterial and fungal species. In comparison with biofilm cultivation directly in microtiter plates, a key advantage of peg lids is the ability to detach pegs for pre-exposure control measurements and for microscopy. Although peg lids are a more expensive substrate for biofilm cultivation than microtiter plates, this approach eliminates concerns that aggregation may be linked to sedimentation of the microorganisms in test wells. Peg lid biofilm reactors are not prone to contamination. For instance, the microtiter plate method of cultivation has been used to culture biofilms of different organisms in each row of the device without any detectable cross-contamination between wells. An assessment of biofilm growth on peg lids indicates that this method of batch culture produces biofilms of reproducible cell density.

Experiments designed to examine biofilm antimicrobial susceptibility using peg lids have two phases, namely, (i) calibration of the equipment and biofilm growth conditions and (ii) high-throughput screening (FIG. 1). Calibration of the peg lid biofilm reactor and optimization of growth conditions for the test organism may involve some effort; however, once the instruments are set up, susceptibility determinations are rapid. The following instructions provide a technique to assess biofilm sensitivity to a twofold dilution gradient of two antimicrobial agents. This approach uses a challenge plate configuration analogous to the standard broth microdilution MIC test¹. In practice, however, the challenge plate can have any configuration, and additional single and combination antimicrobial agents could be tested as desired. After exposure, the surviving biofilm microbes are recovered and microbicidal end points can be determined qualitatively by looking for visible growth in the recovery medium after a suitable period of incubation. Alternatively, immediately after exposure, the surviving microbes can be plated out for viable cell counts (VCCs) and survival can be assessed quantitatively using mathematical analysis. If the experimental design requires biofilm resistance and tolerance to be distinguished from one another, then quantitative susceptibility testing should be performed using two different exposure time periods. In short, the suggested protocol may be followed, and on the basis of experimental results, certain parameters can be optimized to suit a specific experimental design or organism.

These and other aspects of the invention will be made apparent in the figures, description, and claims that follow.

### BRIEF DESCRIPTION OF THE DRAWINGS

FIG. 1 is a flow chart and timeline for biofilm cultivation and susceptibility testing in accordance with the present invention.

FIG. 2 shows an example of a biofilm growth and formation process of the present invention.

FIG. 3 shows an example of a biofilm susceptibility assay of the present invention.

FIG. 4 shows an example of a process for recovering intact biofilm in accordance with the present invention.

FIG. 5 shows an example of a process for establishing MBEC and MIC determinations in accordance with the present invention.

FIG. 6 shows the configuration of a challenge plate used in Example 10.

FIG. 7 is a chart of the MIC, MFC, and MBEC values determined biofilms.

FIG. 8(a-c) illustrates reading qualitative end points from patterns in recovery plates and interpreting biofilm survival data from kill curves.

### DETAILED DESCRIPTION OF THE INVENTION

The invention comprises improved methods and devices for the selection of one or more active agents, either alone or in combination, effective against one or more biofilms alone or in combination. The invention may further comprise optimizing the method and/or devices for growing the biofilm. The invention may further comprise susceptibility testing one or more microorganisms or mixtures of microorganisms, providing measures of resistance and/or tolerance of the microrganism(s).

In some embodiments of the invention, the methods and devices involve setting up and/or calibrating a biofilm growth device, said biofilm growth device comprising a lid comprising at least one peg; optimizing the methods and devices to promote biofilm growth, and susceptibility testing one or more microorganisms.

The methods and devices of the previous two paragraphs may further comprise one or more of the following, alone or in various combinations: optimizing the device and growing conditions specific for a particular microorganism; growing multi-species biofilms; susceptibility testing multi-species biofilms; growing biofilm to an amount greater than about 10⁴ cells; optimizing and/or changing the surface attributes of the peg or substrate to promote biofilm growth and/or cell adherence; evaluating microbial growth in the biofilm reactor, including but not limited to using viable cell count (VCC), determining the number of cells growing in the planktonic inoculum; determining the number of cells growing in the peg biofilms, and assessing the biofilm growth for nonequivalent or asymmetric growth patterns; pre-exposure control measurements; promoting reproducible cell density; biofilm recovery using sonication; qualitative biofilm recovery; quantitative biofilm recovery; optimizing sonication time; establishing end points, including but not limited to measurements for tolerance, measurements for resistance, MIC, MBEC, MBIC, MBC, and MLC; providing a growth medium suitable for the specific microorganism(s); providing a recovery medium suitable for the specific microorganism(s); providing susceptibility testing suitable for the specific microorganism(s); selecting a processing temperature suitable for a specific microorganism's growth, recovery, and/or susceptibility testing; selecting a cultivation time suitable for a specific microorganism's growth, recovery, and/or susceptibility testing; selecting an inoculum amount suitable for a specific microorganism's growth, recovery, and/or susceptibility testing; and selecting a nutritional medium suitable for a specific microorganism's growth, recovery, and susceptibility testing.

One skilled in the art will recognize that the devices and parameters for growing, recovering, and/or susceptibility testing one or more biofilms may involve tailoring the device and parameters for a specific biofilm(s), and further, that this tailoring may include a wide variety of variables. Some of these variables are noted above; other variables are shown in the Examples. These and other variables are included within the scope of the present invention.

An embodiment of the invention includes establishing optimized devices and process parameters for each of the 65+ microorganisms shown in Example 29.

In preferred embodiments of the invention, the devices and methods may be used in susceptibility testing one or more biofilms alone or in combination; and/or in the treatment of infections or conditions mediated by one or more gram-negative bacteria. In the most preferred embodiments of the invention, the devices and methods may be used as a diagnostic tool to determine various compositions, including the optimum composition, for treating one or more biofilms and/or one or more disease or conditions mediated by the biofilm. In some embodiments of the invention, the methods and devices provide diagnostic or clinical susceptibility testing, and in the most preferred embodiments, provide any combination of MBEC, MBC, and MIC values in a single experiment.

The invention also provides methods and devices for selecting one or more biofilm agents, alone or in combination, for the treatment of one or more gram-negative bacteria, alone or in combination; one or more gram-positive bacteria, alone or in combination; one or more diseases or conditions mediated by a gram-negative bacteria; one or more fungal organisms, alone or in combination; one or more diseases or conditions mediated by a gram-positive bacteria; and one or more diseases and/or conditions mediated by fungal microorganisms. As used herein, alone or in combination includes a single microorganism on a single peg or in a single well; a single microorganism alone on a single peg or in a single well on a lid or bottom having a different microorganism in a different peg or well (i.e., multiple microorganisms on a single lid or bottom); and multiple microorganisms on a single peg or well. Thus, in accordance with the present invention, the devices and methods may include single species or multiple species; the multiple species may include a combination lid, plate, or susceptibility test, “combination” referring to a single species separated from another species, but on a single lid or plate. Combination lids, plates, and tests also refers to pre-determined groups of microorganisms grown or tested on a single lid or plate, e.g., a gram (−) lid or a gram (+) lid, or mixtures thereof Multiple species also includes a “mixed” lid, plate, or susceptibility test, “mixed” referring to more than one species in the same peg or well (e.g., a mixed biofilm).

The invention also provides methods and devices for treating one or more diseases or conditions mediated by one or more gram-negative bacteria.

As used herein, gram-negative biofilm or bacteria refers to any bacterium or biofilm formed by that bacterium that is termed gram-negative by one skilled in the art. Typically, gram-negative refers to the inability of a type of bacterium to resist decolorization with alcohol after being treated with crystal violet (Stedman's Medical Dictionary, 28^{th} Ed., 2006). Exemplary gram-negative bacterium families include, but are not limited to E. coli, Burkholderia spp, Acinetobacter spp, Proteus spp, Salmonella spp. Stenotrophomonas spp and Pseudomonas spp, Vibrio, Yersinia, Campylobacteria. Exemplary strains within these families include but are not limited to A. lwoffii, A. radioresistens, A. baumanii, A. heamolyticus, A. calcoaceticus anitatus; E. coli, E coli strain 0157:H7; B. cepacia; P. aeruginosa, Proteus mirabilis, Proteus vulgaris, Stenotrophomonas maltophilia, Yersinia enterocoloticia, Campylobacter jejuni, and Vibrio cholerae.

The invention also provides methods and devices for selecting an antibiotic or combination of antibiotics for the treatment of CF in a specific patient. The present invention also includes methods and devices for treating a patient or subject having a disease or condition mediated or caused by a biofilm. In these embodiments of the invention, a biological sample from a patient or subject is processed with an apparatus or portion of an assembly adapted and/or configured to promote biofilm growth. The biofilm may then be processed with an apparatus or portion of an assembly adapted and/or configured to expose the biofilm to one or more antimicrobial agents or one or more anti-biofilm agents.

The methods and devices or assemblies of the present invention comprise optionally calibrating the equipment; growing the biofilm, preferably including optimizing the apparatus or a portion thereof, and/or optimizing the growth conditions; removing intact biofilm from the growth assembly; and subjecting the biofilm to antimicrobial susceptibility testing, preferably including optimizing the apparatus or a portion thereof, and/or optimizing the exposure conditions in a manner specific for the particular organism(s).

Exemplary biofilm formation devices, biofilm susceptibility devices, and biofilm testing assemblies are described in U.S. Ser. No. 11/996,478 (filed 19 Aug. 2008).

An embodiment of the invention includes an assembly comprising one or more plates pre-loaded with one or more pre-selected anti-biofilm agents against a specific biofilm or biofilms, said plates may be used to identify efficacious individual or combined active agents for treating biofilm-mediated diseases or conditions.

In some embodiments of the invention, the method may also include one or more of the following: growing multiple or plural biofilms under conditions that promote the production of substantially uniform biofilms; screening the biological sample against a large group of active agents; selecting a subgroup of active agents; loading an assay device with multiple or plural active agents in the subgroup; growing biofilm from a specific patient's or subject's sample; screening the biofilm from the specific patient or subject against the subgroup of active agents; reading the results; determining the appropriate active agent or combination of active agents suitable for the particular biofilm; conducting a turbidity assay if the microorganism produces visible turbidity when growing (e.g. Pseudomonas); and conducting a plating assay if the microorganism does not grow with visible turbidity.

An embodiment of the invention includes methods for selecting specific combinations of antibiotics that have efficacy against isolates of one or more gram-negative bacteria as a biofilm by screening a broad range of clinical isolates of a species against an extensive panel of antibiotics alone or in combination to identify combinations with efficacy against biofilm grown organisms.

An embodiment of the invention includes determining the active agent or antibiotic(s) of choice for the treatment of a biofilm infection by challenging the biofilm of the patient's specific isolate against the diagnostic plate specific for the species that forms the biofilm.

An embodiment of the invention includes rehydrating a species specific plate of preloaded antibiotics as the challenge plate to identify antibiotics with efficacy against the specific pathogen. Plates may be frozen (no rehydration required), or lyophilized, freeze dried or vacuum dried.

An embodiment of the invention includes a well plate containing frozen or lyophilized antibiotic combinations that can be re-hydrated to be used in an antibiotic susceptibility assay.

An embodiment of the invention includes growing biofilm obtained from a biological specimen obtained from a patient, and using the biofilm in a susceptibility assay. In this embodiment of the invention, the susceptibility assay provides which active agent or combination of active agents is best suited to eradicate a gram-negative Pseudomonas species or a Pseudomonas aeruginosa biofilm. In this embodiment, the susceptibility assay may also provide which active agent or combination of active agents is best suited to treat a disease or condition mediated by the gram-negative biofilm. An embodiment of the invention includes challenging a biofilm against selected combinations of an anti-microbial or an anti-biofilm agent, thereby identifying the most appropriate combination. Some embodiments of the invention further include using the identified antimicrobial agent or agents to treat a patient, to treat a microorganism, and/or to change an existing treatment regimen or antimicrobial agent to a more medically beneficial regimen or agent(s).

An embodiment of the invention includes providing MBEC values in the diagnosis and treatment of any gram-negative microorganism capable of biofilm formation, and using those values to treat or develop a treatment protocol for any gram-negative microorganism-mediated disease, infection, or condition. The invention may further include providing MIC and/or MBC values.

In a further aspect of the invention, after growing the biofilm on adherent sites on a lid or plate, the methods and devices may include dislodging the biofilm from the biofilm adherent sites and further incubating the biofilm. Dislodging the biofilm from the biofilm adherent sites may include dislodging the biofilm from each biofilm adherent site into a separate well of a microtiter plate or base. In preferred embodiments of the invention, the biofilm is dislodged using any process that results in intact biofilm being removed from the adherent sites. The inventors have found that using centrifugation removes only a portion of the microorganism, and therefore any resulting assay may be incomplete or inaccurate.

Preferably, the plural biofilm adherent sites are formed in plural rows, with plural sites in each row; and the container includes plural channels, with one channel for each row of plural biofilm adherent sites. Devices or assemblies so configured permit high throughput analysis of the biofilm.

An embodiment of the invention also includes a pharmaceutical composition suitable for treating one or more gram-negative bacteria, and/or one or more diseases or conditions caused by the gram-negative bacteria. In these embodiments of the invention, the pharmaceutical composition includes one or more active agents specifically chosen as effective. In these embodiments of the invention, the active agent(s) are selected by processing a biological sample from a patient through biofilm growth and susceptibility testing devices of the present invention. These device(s) grow biofilm from the bacteria found in the patient's sample, then subject the biofilm to a panel containing at least one active agent. The optimum active agent or combination of active agents may then be selected for use in treating the patient.

A biofilm reactor, as used herein, comprises a lid having one or more substrates, wherein said lid is configured to engage a bottom plate. The substrate may be variously configured, but is typically a peg or the like. The first bottom plate may be variously configured, including but not limited to a typical microtiter plate having a well configured to receive an individual peg; or a trough having one or more channels configured to receive at least one peg. It is intended that the lid and first bottom plate are configured to promote biofilm growth. In preferred embodiments of the invention, the lid/bottom assembly that comprises a biofilm reactor exhibits reduced or eliminated contamination.

It is intended that the lid and/or pegs may be configured to engage at least one second bottom plate. It is intended that the lid and second bottom plate may be variously configured to provide and/or promote susceptibility testing.

Peg lids, as used herein, refers to the lid noted above, suitable for growing and testing one or more biofilms. Suitable, as used here, refers to various structures and characteristics, including but not limited to a peg detachable from the lid, breakable or removable pegs, pegs that have been scored so that they may be removed from the lid; pegs that are positioned in the lid with a permanent or removable adhesive backing; a coated or uncoated substrate or peg; and/or a substrate or peg comprising or coated with any of a wide assortment of materials that promote biofilm growth and/or recovery. The preferred peg lid comprises polystyrene, but may be formed of any material or materials that have a neutral electrostatic charge. Peg lids may be constructed individually, or are commercially available from Nunc, Trek Diagnostics, and other manufacturers. Commercially available structures may need to be altered or reconfigured in accordance with the teachings of this invention to provide biofilm growth, promote biofilm growth, provide and/or promote biofilm adherence; provide and/or promote biofilm recovery; and provide and/or promote biofilm susceptibility testing.

Exemplary parameters and controls for biofilm cultivation on peg lids. Reactor set up and growth conditions. Microbes depend on diverse environmental and nutritional cues to attach to a surface and to initiate biofilm formation. Fastidious criteria for microbial surface attachment can be met by mixing and matching a set of reactor parts and by coating pegs with conditioning films. Other considerations include the rate of motion of the inoculated reactor, incubation temperatures and time periods, inoculum size, atmospheric gases, composition of the growth medium and frequency of medium exchange. We have conducted a comprehensive review of the growth conditions reported in the literature for growing biofilms on peg lids, and all of these discrete parameters can be thought of as adjustable steps in a core protocol (Example 28). To date, variations in this core procedure have been used to cultivate biofilms representing >65 different microbial species (Example 28), of which several have been grown in multispecies biofilms.

There are several considerations when growing biofilms on peg lids:

Surface attributes may be important for getting microbes to attach to a substratum. Lids can be made from different materials, such as raw polystyrene (the MBEC assay) or from chemically modified plastics, such as those used for solid-surface enzyme-linked immunosorbant assays (Nunc Immuno-TSP). It is also possible to coat peg lids with conditioning films to facilitate the adhesion of fastidious microorganisms that might not otherwise stick to the surface. Such coatings might include L-lysine²⁰, BSA, trichloroacetic acid treated with ethylene oxide²¹, human saliva^{22,23} and polycyclic aromatic hydrocarbons, such as phenanthrene²⁴.

In addition to microtiter plates, peg lids can be fit into troughs and these platforms can be used for biofilm cultivation on an orbital shaker or a rocking table. A disadvantage of using the trough method is that a rocking table is not a customary piece of equipment in many microbiological laboratories. However, not all microbial species will form biofilms with consistent peg-to-peg cell densities on an orbital shaker (or vice versa), and therefore, the choice of platform is dictated by the requisite growth conditions for the microorganism²⁵. We would recommend the use of the microtiter plate method for biofilm cultivation as a first choice over the trough format of the assay because of its increased simplicity.

Incubation temperatures and time periods not only depend on the growth optima of the test organism but also can be influenced by temperature-dependent changes in production of extracellular polymers or adhesins. For example, certain Escherichia coli strains produce cellulose and curli fimbriae at 23° C. but not at 37° C., and thus a temperature shift can affect the adherence of E. coli to a surface²⁶.

Similar to MIC testing, inoculum size for biofilm cultivation is measured using McFarland standards; however, as this is based on optical density (OD) measurements, these standards can represent different numbers of cells for different organisms. Relatively lower starting inoculum sizes have been linked to increased biofilm production for some bacteria, such as for Pseudomonas aeruginosa PAO1 (ref. 27) (Example 28). By contrast, a relatively larger inoculum size seems to be essential for biofilm formation by other species, such as for Rhizobium leguminosarum biovar viciae (ref 28).

Atmospheric gases can be controlled in air-tight environmental chambers or incubators to facilitate biofilm formation by facultative and obligate anaerobes

Electron acceptors, host factors, carbon and nitrogen sources, as well as inorganic ions, such as magnesium and phosphorous, can be environmental cues that affect microbial adhesion and growth on surfaces². It is because of this biological fact that there can be no universal medium for growing a biofilm, even among microorganisms that can be routinely cultured on rich laboratory media. Similar to larger scale reactors, the best experimental approach is to choose a medium that most closely resembles the environment of interest.

We recommend the experimenter start with conditions suited, appropriate, or advantageous to the specific species being tested. Exemplary conditions are listed in Example 28 to cultivate the organism of interest. If the test organism has not been grown on peg lids before, a good starting point is to use a nutritional medium that is known to support growth of the microbe in vitro. Optimization of biofilm growth for more fastidious organisms can be achieved by experimenting with different reactor assemblies, medium formulations, surface coatings and other parameters as seen fit, and then by testing them empirically. For instance, it might be possible to test several growth media for their ability to promote biofilm formation on pegs by inoculating different media with the same test organism. These inocula could be arranged in separate wells of a microtiter plate and a single peg lid could be used as the substratum. The number of cells in biofilms could then be quantified using the methods presented in the core protocol.

Media for susceptibility testing and cell recovery. Growth media for susceptibility testing. The guidelines set by the Clinical Laboratory Standards Institute (CLSI) and the European Committee on Antimicrobial Susceptibility Testing indicate that in most cases, standardized bacterial MIC testing should be performed in cation-adjusted Mueller-Hinton broth¹. Standardization of the test medium has been essential for interlaboratory reproducibility of MIC testing. No such standard medium exists, however, for biofilm susceptibility testing, and this is likely because of the different nutritional requirements for getting biofilms to grow under laboratory conditions. When choosing a medium for microbial exposure, it is most important that it is chemically compatible with the test agent and that it contains no components that might detrimentally affect biofilm growth. For example, polysorbate-80, an additive routinely used to prevent the adsorption of some antibiotics to plastic surfaces, can inhibit biofilm formation by some Staphylococcus³⁰ and Pseudomonas³¹ sp., and therefore, should not be used during biofilm susceptibility testing. A good experimental strategy is to choose a test medium that most closely resembles the environment in which the biofilm is likely to encounter the antimicrobial agent that is being tested.

Sonication and biofilm cell recovery. Biofilm cells can be recovered using low-frequency (60 Hz) vibrations to disrupt cells into a rich medium that contains 1% Tween-20 or a comparable surfactant. As an exception, recovery of biofilm Clostridium difficile

(Example 28) requires 0.5% Tween-80 supplemented with 0.1% taurocholate in the recovery medium. This process is carried out at room temperature (20-25° C.) and the recovered cells are immediately serially diluted and plated onto agar in less than the doubling time of the test organism. This ensures that there are no artificial increases in biofilm cell numbers due to processing time. Vibrations can be generated using a water table sonicator, wherein the peg lid, which is inserted into a microtiter plate containing the recovery medium, is placed on the steel insert tray of this device. Ali et al.³² recommend a sonication time of 10 min, as shorter time periods led to incomplete cell recovery and longer time periods (i.e., 15 min) did not result in significantly increased cell recovery from pegs. For example, Listeria innocua grown in the CBD using the parameters listed in Example 28 yielded 5.4±0.1, 5.9±0.1 and 6.0±0.1 log₁₀ colony forming units per peg (CFU per peg) with 5, 10 and 15 min of sonication, respectively. Ali et al.³² recommended this intermediate sonication time as recovery was high and consistent; moreover, an intermediate sonication time reduced the possibility of damage to injured cells that extended time periods might cause, especially after susceptibility testing. Nonetheless, one might find it worthwhile testing this optimum sonication time for different instruments and organisms. A simple strategy here would be to follow the protocol and to test different sonication time periods for an effect on the mean VCC determined from batch culture biofilm growth controls for the desired test organism.

Inactivating antimicrobial agents. In general, there are three optional methods to inactivate antimicrobials: (i) membrane filtration, (ii) dilution of the agent to a sub-inhibitory level and (iii) the addition of a neutralizing agent³³. In the core protocol for biofilm susceptibility testing, we opt to dilute the antimicrobial agent back to sub-inhibitory levels by rinsing the biofilms twice before disrupting the cells into the recovery medium. If the experimental design is modified to include a comparison of biofilm and planktonic cell susceptibility, then biofilms and planktonic cells could be treated with a neutralizing agent. In this way, similar inactivating regimens can be used to carry out a fair comparison of biofilm and planktonic cell susceptibilities. If carry-over of low antimicrobial concentrations prevents accurate VCCs, or if the susceptibility data are to be used for a regulatory submission³⁴, then use of a neutralizing agent in addition to rinsing is warranted. Neutralizing agents, in general, are filter sterilized and then added to the sterile recovery medium at an appropriate molar concentration that typically exceeds the working concentration of the antimicrobial agent. A list of neutralizing agents for antibiotics, biocides and some metal ions can be found elsewhere^{33,34}. It is important to confirm that the neutralizer works and does not harm the recovered microorganisms, and for a discussion of this we suggest that one should consult the guidelines published by the American Society for Testing and Materials (ASTM International)³⁵.

In addition to susceptibility testing, peg lid biofilm reactors of the present invention serves as the starting point for a variety of downstream applications; e.g. additional or alternative modifications, applications and limitations:

1) Biofilm biomass may be stained on peg lids with crystal violet^{40,41}, which is adapted from the O'Toole and Kolter^{15,16} method of staining biofilms grown in the wells of microtiter plates.

2) Biofilm structure on pegs may be determined by microscopy^{7,25,42,43}; however, biofilms cultivated on pegs are subject to complex fluid dynamics and, although gross morphological changes in structure may be discerned, flow cell models might be more suitable for testing this. Batch culture systems, such as the peg lid biofilm reactor described here, do not provide continuous flow or replacement of media and therefore may significantly influence the structure of the biofilm. Thus, the intricate microcolony structure of biofilms obtained using flow cells might be altered or absent from peg lid biofilms. Even so, it is possible to visualize 3-D patterns in peg lid biofilm killing by antibiotics that are similar to those produced in flow cells.

3) Low-speed centrifugation can be used to disrupt cells from pegs into a recovery medium⁴⁴.

4) RT-PCR and promoter-reporter constructs can be used to measure the gene expression in biofilms; however, the tiny amount of biomass produced on each peg makes the peg lid biofilm reactor ill-suited for proteomics.

5) Cell viability may be assessed using a variety of methods, including quantitative PCR^{22,23} and tetrazolium salts^{42,43}.

6) Challenge plate configurations can be set up to screen libraries of compounds for anti-biofilm activity, to perform checkerboard assays to identify antimicrobial antagonism or synergy¹⁹ and to perform multiple combination susceptibility testing¹³.

7) Isogenic mutants at similar biofilm cell densities can be compared to determine differences in antimicrobial sensitivity due to gene deletion or overexpression^{45,46}.

8) It is possible to modify this technique to determine MIC data and VCCs for planktonic cells shed from the surface of the biofilm, while simultaneously determining biofilm susceptibility. An important limitation of this approach is that the starting number of cells for planktonic susceptibility testing cannot be determined, and thus, log-killing of planktonic cells cannot be calculated. Nonetheless, MIC measurements made using this type of experimental design in some instances approximate those made using a standard CLSI MIC test¹⁷. Moreover, planktonic cells that are shed from the surface of the peg biofilms and isolated from the wells of the challenge plate have a different sensitivity to antibiotics and metal ions than the biofilms from which they were derived^{47,48}. This nonstandardized method to test planktonic cell susceptibility is not presented here, and instead we direct researchers to a discussion of this approach elsewhere^{17,47,49}.

Peg lids (MBEC P&G or HTP assays, Innovotech or Nunc Immuno-TSP, Nunc, cat. no. 445497) The MBEC and Nunc Immuno-TSP peg lids are manufactured in different ways. The MBEC peg lid is designed as a substratum for biofilm growth. These lids are made from polystyrene, bare an overall neutral electrostatic charge and have a plastic backing, as well as are engineered with break points that facilitate detachment of individual pegs. The MBEC P&G assay is packaged with a microtiter plate, whereas the HTP assay comes with a trough that serves as the inoculum reservoir. In contrast, the Nunc Immuno-TSP lids were designed as supports for solid-surface enzyme-linked immunosorbant assays, but can also be used for biofilm cultivation. These lids have a chemically modified polystyrene surface, bare an overall positive electrostatic charge and lack the plastic backing and break points that facilitate peg detachment. These lids are packaged with a trough that can be used as an inoculum reservoir, but this can be swapped for a microtiter plate at ones discretion. Nunc Immuno-TSP lids will need to be modified for biofilm assays as described in the Examples.

Building and sterilizing peg lid reactors If not carried out by the manufacturer (e.g., Nunc-TSP lid), trim an adhesive backing (e.g., Costar plate sealers) and fit it to the top of the peg lid. This will maintain sterility of the device once pegs have been removed for control measurements. It is also possible to swap the troughs that come with the Nunc-TSP lids for microtiter plates at this point. If peg lids are nonsterile when purchased from the manufacturer, if an adhesive backing has been applied before use or if the reactors have opened and parts have been swapped, assemble the device, seal it in an air-tight plastic bag and sterilize it using ethylene oxide (Anprolene), according to the directions of the supplier.

Sterile agar and broth growth media specific for the microorganism to be cultured. There are no standardized media for biofilm cultivation or susceptibility assays; however, there must be no variation in the chosen medium composition from one experiment to the next. This will ensure reproducibility of intra- and interlaboratory results. One is cautioned to strictly control the composition of the growth medium when comparing data sets generated by technicians in the same and different laboratories

A device of the present invention may comprise a biofilm growth assembly 1, a biofilm challenge assembly 2, a rinsing assembly 3, and a biofilm dislodging and re-growth assembly 4. Used in concert, the assemblies provide MIC, MBC, and MBEC values in a single experiment.

In accordance with the present invention, the biofilm growth assembly 1 may include a base or plate 20 configured to receive a lid 10. Lid 10 may be configured to include one or more projections 12 that extend into a space defined by base 20. In most preferred embodiments of the invention, the biofilm growth assembly 1 is rocked, moved, or the like so that the growth fluid in the assembly flows or moves across projections 12. In preferred embodiments of the invention, base 20 is an incubation base and is configured to provide each projection with substantially equivalent exposure to the source of microorganisms and its nutrient/growth broth.

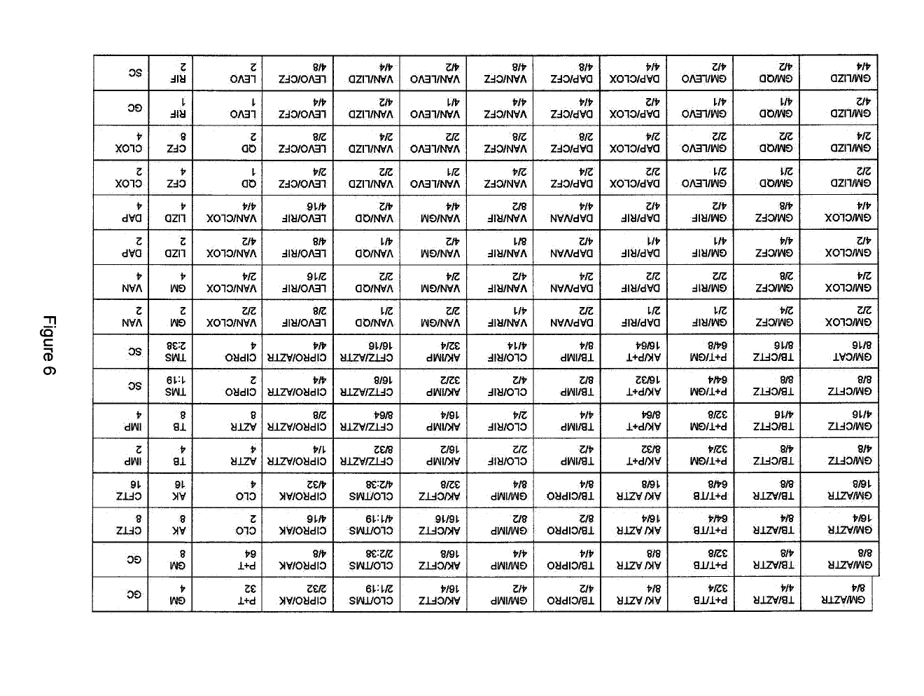

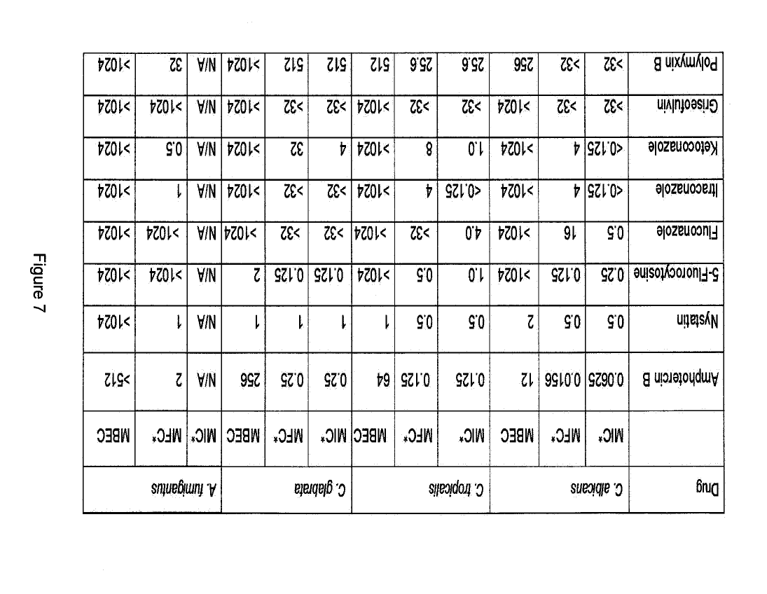

In accordance with the present invention, the biofilm challenge assembly 2 comprises a second base or plate 21 configured to receive a lid 60 having projections 61 typically covered by biofilm. Projections 61 extend into one or more wells configured in plate 21. A typical second base 21 is a standard 96 well microtiter plate, although one skilled in the art will readily recognize that other configurations may be used. Second base 21 includes one or more anti-biofilm agents in the wells. In accordance with the present invention, second plate 21 may be removed and used for determining the MIC value of the non-biofilm (e.g., planktonic) microorganism (see FIG. 5).

In accordance with the present invention, the biofilm rinsing assembly 3 comprises a third base or plate 40 configured to receive a lid 60 having projections 61 typically covered by biofilm. Projections 61 extend into one or more wells configured in plate 40. A typical third plate 40 is a standard 96 well microtiter plate, although one skilled in the art will readily recognize that other configurations may be used. Third plate 40 includes one or more rinsing and/or neutralizing agents in the wells.

After rinsing, lid 60 may then be joined with a fourth base 50, also referred to as a recovery plate. Lid 60 and fourth base 50 form the biofilm disruption assembly 4. The recovery plate contains recovery media, and, in accordance with the present invention, assembly 4 may be subjected to sonication and biofilm re-growth (confirming that the biofilm has not been removed). In preferred embodiments of the invention, the recovery medium includes one or more neutralizing agents. As shown in the examples, assaying the projections on lid 60 after it has been exposed to recovery media provides an MBEC value of the microorganism, and plating from the recovery plate provides an MBC value.

The device includes biofilm lid 10 composed of tissue grade plastic or other suitable material (e.g. stainless steel, titanium) with projections 12 extending downwardly from the lid 10. The projections 12 may be biofilm adherent sites to which a biofilm may adhere, and may be configured into any pattern or shape suitable for use in conjunction with a channel or well-containing bottom, such as base 20. The pattern of projections 12 preferably mirror the pattern of channels and/or wells in convention plates, e.g. a 96-microtiter or well plate commonly used in assay procedures. In most preferred embodiments of the invention, the projections 12 are preferably formed in at least eight rows 14 of at least twelve projections each. Other numbers of rows or numbers of projections in a row may be used, but this is a convenient number since it matches the 96 well plates commonly used in biomedical devices. Additional or some of the projections as shown may be used to determine the initial biofilm concentration after incubation. The exemplary projections 12 shown are about 1.5 cm long and 2 mm wide, but may be any size and/or shape.

The lid 10 has a surrounding lip 16 that fits tightly over a surrounding wall 28 of the vessel 20 to avoid contamination of the inside of the vessel during incubation.

Base 20 serves two important functions for biofilm development. The first is a reservoir for liquid growth medium containing the bacterial population which will form a biofilm on projections 12. The second function is having a configuration suitable for generating shear force across the projections. While not intending to be limited to any particular theory of operation, the inventors believe that shear force formed by fluid passing across the projections promotes optimal biofilm production and formation on the projections.

Shear force on the projections 12 may be generated by rocking the vessel 20 with lid 10 on a tilt table 30. The inventors have found that using a rocking table that tilts to between about 7° and about 11° is suitable for most applications. In preferred embodiments of the invention, the rocking table should be set on about 9°. It is intended that the invention should not be limited by the use of an actual degree of tilt, but that any tilt used for any particular machine be appropriate for growing biofilm in accordance with the present invention.

The projections 12 may be suspended in the channels or wells so that the tips of the projections 12 may be immersed in liquid growth medium flowing in the channels. The ridges 26 channel the liquid growth medium along the channels 24 past and across the projections 12, and thus generate a shear force across the projections. Rocking the vessel 10 causes a repeated change in direction of flow, in this case a repeated reversal of flow of liquid growth medium, across the projections 10, which helps to ensure a biofilm of equal proportion on each of the projections 12 of the lid 10. Rocking the vessel so that liquid flows backward and forward along the channels provides not only an excellent biofilm growth environment, but also simulates naturally occurring conditions.

Each projection 12 and each channel 24 preferably has substantially the same shape (within manufacturing tolerances) to ensure uniformity of shear flow across the projections during biofilm formation. In preferred embodiments of the invention, channels 24 should all be configured or connected so that they share the same liquid nutrient and bacterial mixture filling the basin 22. The inventors have found that substantially uniform channel configuration and access to the same source of microorganisms promotes the production of an equivalent biofilm on each projection, equivalent at least to the extent required for testing anti-biofilm agents. Biofilms thus produced are considered to be uniform. Results have been obtained within P<0.05 for random projections on the plate.

Sensitivity of a biofilm may be measured by treating the biofilm adherent sites with one or more anti-biofilm agents, i.e., challenging the biofilm, and then assaying the biofilm. This may be accomplished by placing the lid 60 (having a biofilm formed on the projections) into a second base 21 adapted to receive lid 10 and projections 12. In preferred embodiments of the invention, lid 60 engages second base 21 in a manner sufficient to prevent contamination of the assembly. As used herein, a manner sufficient to prevent contamination refers to the configuration and assembly of mating structures so that the contents of the closed assembly are free of outside contamination.

In accordance with the present invention, one skilled in the art may use any arrangement or scheme for challenging a group of biofilms. For example, all of the wells of the challenge plate may include the same anti-biofilm agent; plural or multiple wells may include different doses of the same anti-biofilm agent; plural or multiple wells in a single row may include the same dose or different doses of anti-biofilm agent; plural or multiple rows may include the same dose or different doses of anti-biofilm agent; plural or multiple wells or plural or multiple rows may include more than one anti-biofilm agent; or plural or multiple wells or plural or multiple rows may include more than one anti-biofilm agent, varying the dose by well, by row, and/or by anti-biofilm agent. It is intended that the configuration and arrangement of wells, type and number of anti-biofilm agents, and dose in each well should be variable as desired by one skilled in the art to achieve a specific purpose, e.g., testing one or more biofilms with one or more anti-biofilm agents using as many variables as reasonable to the intended purpose.

For example, projections 12 that have been incubated in the same channel 24 of the vessel 20 may be treated with a different anti-bacterial reagent. In this manner, consistent results may be obtained since the growth conditions in any one channel will be very similar along the entire channel and thus for each projection 12 suspended in that channel. This helps improves the reliability of treatment of different projections 12 with different anti-bacterial reagents. The examples show different arrangements suitable for use with the assemblies of the present invention.

As noted above, a device of the present invention may be loaded with one or more anti-biofilm agents. An incomplete and exemplary list of possible anti-biofilm agents include, but are not limited to: Antibiotics. Including, but not limited to the following classes Aminoglycosides; Antipseudomonals, including Cephalosporins; beta.-Lactams; Antibiotics; Urinary Tract Antiseptics, such as Methenamine, Nitrofurantoin, Phenazopyridine and other napthpyridines; Penicillins, Tetracyclines; Tuberculosis Drugs, such as Isoniazid, Rifampin, Ethambutol, Pyrazinamide, Ethinoamide, Aminosalicylic Acid, Cycloserine; Anti-Fungal Agents, such as Amphotericin B, Cyclosporine, Flucytosine Imidazoles and Triazoles Ketoconazole, Miconazaole, Itraconazole, Fluconazole, Griseofulvin; Topical Anti Fungal Agents, such as Clotrimazole, Econazole, Miconazole, Terconazole, Butoconazole, Oxiconazole, Sulconazole, Ciclopirox Olamine, Haloprogin, Tolnaftate, Naftifine, Polyene, Amphotericin B, and Natamycin.

Several different conventional methods may be used to count the bacteria. It may be done by incubating the sonicated bacteria, taking serial dilutions and visually counting the colony forming units, or automated methods may be used, as for example using an optical reader to determine optical density. It has been found however that the optical reader of turbidity is too imprecise for practical application, and it is preferred that vital dye technology be applied to automate the measurement of viability, by treating the biofilm with a vital dye, and measuring the intensity of light given off by the dyed biofilm. In the case of using vital dye technology, the biofilm need not be further incubated. One skilled in the art will recognize that other dyes for cell mass may be used; these dyes may be later extracted and read for OD (a measure of remaining cell biomass). In a further embodiment, the assay may be carried out by sonicating the cells until they lyse and release ATP and then adding luciferase to produce a mechanically readable light output. In a still further embodiment, the assay may be carried out directly on the biofilm on the projections using a confocal microscope, although it should be considered that this is difficult to automate. In the examples that follow, the results are obtained from a manual count following serial dilution.

The concentration (MBEC) of anti-bacterial reagent at which the survival of bacteria or biofilm falls to zero may be assessed readily from the assay. Likewise, the MIC may also be determined from the assay.

The inventors have found that in some instances a biofilm will not form without the inclusion of host components in the biofilm. Host components may therefore be added to the growth medium in the vessel during incubation of the bacteria to form the biofilm. Host components that may be added include serum protein and cells from a host organism. For the testing of the effect of different host cells and components, the ends of the channels 24 may be sealed by walls to prevent growth medium in one channel from flowing into another, thus isolating the bacteria growth in each channel from other channels. The device thus described may also be used to test coatings used to inhibit biofilm growth and to test coatings which may enhance biofilm formation. In an initial step, the projections 12 may be coated with a coating to be tested, and then the biofilm grown on the projections. The biofilm may then be assayed, or treated with anti-bacterial reagent and then assayed. The assay may be in situ or after dislodging of the biofilm. Different coatings may be tested on different rows of pegs. Enhanced biofilm formation may be used to create large viable biofilms for biofermentation.

### Definitions

As used herein, assembly refers to an integrated collection of elements or components designed or configured to work in concert. A typical assembly of the present invention includes a lid and its corresponding base or plate. In some embodiments of the invention, an element of one assembly may function or work with a separate assembly. For example, the lid of assembly 1 may be used as the lid in assembly 2, i.e., with a different base. In preferred embodiments of the invention, a lid may engage a base in a removable, sealingly fashion. In other embodiments of the invention, a lid may engage a base in a closed, sealingly fashion; in these embodiments, it may be desirable to adapt other elements of the assembly so that they are removable, e.g., one or more removable projections.

As used herein, challenge plate refers to any base having one, multiple, or plural configurations of wells, troughs, or the like, said plate being used to expose one or more biofilms to one or more anti-biofilm agents. A typical challenge plate may be used to determine biofilm growth in an environment that includes one or more anti-biofilm agents. In a later step of a process of the present invention, the challenge plate may be used to determine the MIC value of any planktonic microorganism. An exemplary challenge plate is shown in FIGS. 3 and 5.

The challenge plate may be used to screen antimicrobial libraries, multiple combination susceptibility testing, and gene deletion or over-expression.

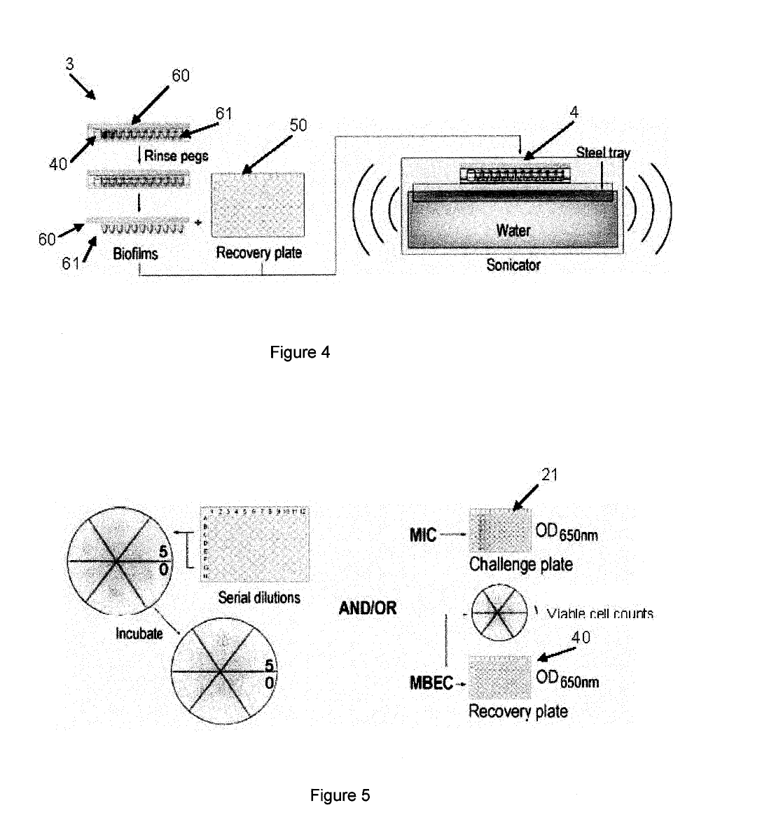

As used herein, recovery plate refers to any base having one, multiple, or plural configurations of wells or the like, said plate being used to rinse biofilm after it has been exposed to an anti-biofilm agent, neutralize any anti-biofilm agent, to collect any disrupted biofilm after the assembly has been sonicated, or combinations thereof. In a later step of a process of the present invention, the recovery plate may be used to determine the MBEC value of any biofilm formed in the process. An exemplary recovery plate is shown in FIGS. 4 and 5.

As used herein, neutralizing agent refers to any composition suitable for reducing or counteracting any toxicity caused by an anti-biofilm agent. A neutralizing agent is appropriate if it is effective for the anti-biofilm agent(s) being used and for a particular biofilm. The choice of neutralizing agent is within the skill of the art. Several neutralizing agents and compositions are shown in the Examples. As described in the Examples, a recovery medium is a composition that includes one or more neutralizing agents.

As used herein, active agent or anti-biofilm agent refers to one or more agents that are effective in reducing, degrading, or eliminating a biofilm or biofilm-like structures. The present invention includes but is not limited to active agents that are already well known, e.g., antibiotics, anti-microbials, and biocides. One or more active agents may act independently; one or more active agents may act in combination or synergistically; one or more active agents may be used sequentially or serially.

As used herein, a panel or library of active agents refers to a collection of multiple or plural active agents grouped according to a pre-determined strategy. For example, a first library may include one or more active agents that show some degree of potential in being effective against a particular biofilm. A second library may begin with a subset of the first library, and is designed to narrow the choices effective active agents, or to provide more information about a particular subset of active agents. A panel or library may also include a proprietary or non-proprietary group of active agents grouped according to a pre-determined strategy, e.g., variable doses.

As used herein a composition containing an active agent may include one or more active agents, and may further include one or more additional agents, including but not limited to bacteriocins or other anti-bacterial peptides or polypeptides, one or more disinfectants or the like, one or more surfactants or the like, one or more carriers, physiological saline or the like, one or more diluents or the like, and one or more preservatives or the like.

As used herein, sample refers to a biological or fluid sample taken from a patient, animal, or environment; sample expressly includes any source or potential source of microorganism. A patient's isolate is derived by standard laboratory methods and prepared for assay using by standard laboratory practice (CLSI). As used herein, biofilm challenge involves the placement of the biofilm culture, grown on a substrate as noted above, into the wells of the challenge plate, thereby exposing planktonic and/or biofilm to a range of concentrations or a spectra of anti-biofilm agents. In preferred embodiments of the invention, the concentration of anti-biofilm agent(s) is selected for its possible effectiveness against the target organism. Incubation time and conditions and medium used will vary with isolate.

As used herein, efficacy is based on the ability of the active agent or active agents to have activity of the biofilm and is defined on the basis of MIC (minimal inhibitory concentration), MBC (minimal biocidal concentration), and MBEC (minimal biofilm eradication concentration). The standard assay for testing the antibiotic susceptibility of bacteria is the minimum inhibitory concentration (MIC), which tests the sensitivity of the bacteria in their planktonic phase. The MIC is of limited value in determining the true antibiotic susceptibility of the bacteria in its biofilm phase. The MBEC, on the other hand, allows direct determination of the bacteria in the biofilm phase, and involves forming a biofilm in a biofilm growth device or plate, exposing the biofilm to one or more test antibiotics or active agents for a defined period, transferring the biofilm to a second plate having fresh bacteriologic medium, and incubating the biofilm overnight. The MBEC value is the lowest active agent dilution that prevents re-growth of bacteria from the treated biofilm.

As used herein, treatment protocol refers to a dose of one of more active agents, the composition of the active agent, and how often it should be administered to a patient. With the devices and methods of the present invention, the treatment protocol can be tailored to a specific human or animal, a specific biofilm or biofilms, and/or a specific disease or condition. For some diseases and conditions, e.g., CF, it may be desirable to perform separate assays at different times to optimize the course of treatment, particularly optimizing treatment or the concentration of active agent(s) over time. For example, it is believed that a CF patient's condition changes over time as both the patient and the infection change; it would be a beneficial result to monitor those changes and alter any treatment as required.

As used herein beneficial result refers to any degree of efficacy against a microorganism or biofilm. Examples of benefits include but are not limited to reduction, elimination, eradication, or decrease in a biofilm or a microorganism that forms a biofilm; and the capability of treating a microorganism hidden or protected by a biofilm. Exemplary examples of a beneficial result in the manner in which a patient is treated includes but is not limited to the ability or capability of treating a specific patient, of the ability to tailor a treatment protocol for a particular patient at a particular time; and of the increased ability of being able to choose a particular active agent or agents. A beneficial result may also include any diagnostic, medical, or clinical benefit or improvement that assists the doctor or the patient in determining the appropriate active agent(s) and/or treatment protocol. For example, beneficial results are obtained when a panel of possible active agents can be tested rapidly, with greater efficiency, and/or with a greater number of combinations.

The potential patient benefits are improvement in quality of life; and the delay in the progression of disease.

The potential doctor benefits are improved patient outcomes; greater confidence in susceptibility testing; reduction of treatment failures; and quantification of combination antibiotic choices.

The potential diagnostic laboratory benefits are reduced susceptibility testing caused by treatment failures and greater confidence in susceptibility testing.

The benefits to the Healthcare system are reduced costs of drug treatment and hospitalization; delay in lung transplantation costs; and reduced resistance development due to the use of inappropriate drugs

As used herein susceptibility testing or similar phrases refers to determining if and by how much an active agent affects the growth or condition of a microorganism in a biofilm. In the devices and methods of the present invention, susceptibility testing is distinguished from prior art methods by using high through-put devices, typically a peg lid device or assembly, by forming a biofilm in a non-static environment, and by generating biofilms through a flow system.

Susceptibility testing, as noted above, may be used to determine one or more of several endpoints, e.g., MBEC, MBIC, MBC, etc. In accordance with the present invention, susceptibility testing does not include MIC or planktonic testing alone; rather, susceptibility testing includes biofilm testing alone, or in combination with planktonic testing.

As used herein, high throughput refers to the capability of growing and/or assaying a high number of biofilms and/or a high number of anti-biofilm agents at the same time or in the same procedure. Typically, high throughput translates into structural elements in one or more of the assemblies in order to increase speed or quantities of materials being grown or tested, e.g., a 96 well assay plate, a top adapted to and configured to engage the 96 well plate, a top with pegs corresponding to the wells, and a biofilm growth plate with channels so that you can process a large number of individual biofilms at the same time.

### Timing

Reagent preparation: 3 h (plus 2 days of drying time for agar media)

Equipment setup: 3 h

Optimization of growth conditions and test for equivalent biofilm growth:

Step 1A, Colony suspension method: <15 min (plus 2 overnight incubations)

Step 2B: 10 min (plus 24 h incubation)

Steps 3-8: 15 min (plus 24 h incubation)

Box 2: 90 min (plus 24 h incubation)

Data entry and calculations: 30 min

High-throughput screening: (for each device)

Steps 1A: <15 min (plus 2 overnight incubations)

Step 2B: 10 min (plus 24 h incubation)

Steps 3-8: 15 min (plus 24 h incubation)

Steps 9-15: 45 min

Steps 16-31: 75 min (plus 24 h incubation)

Steps 32-38: <15 min

Step 39B: 90 min (plus 24 h incubation)

Data entry and calculations: 60 min

As used herein, a pharmaceutical composition is any composition suitable for use in treating a disease or condition involving or mediated by a microorganism. A pharmaceutical composition of the present invention comprises one or more active agents selected by using a susceptibility device described herein. The pharmaceutical composition may also include any other ingredient(s) which one skilled in the art might determine is appropriate or beneficial. Other such ingredients include but are not limited to one or more adjuvants, one or more carriers, one or more excipients, one or more stabilizers, one or more permeating agents (e.g., agents that modulate movement across a cell membrane), one or more imaging reagents, one or more effectors; and/or physiologically-acceptable saline and buffers. Generally, adjuvants are substances mixed with an immunogen in order to elicit a more marked immune response. The composition may also include pharmaceutically acceptable carriers. Pharmaceutically acceptable carriers include, but are not limited to, saline, sterile water, phosphate buffered saline, and the like. Other buffering agents, dispersing agents, and inert non-toxic substances suitable for delivery to a patient may be included in the compositions of the present invention. The compositions may be solutions suitable for administration, and are typically sterile, non-pyrogenic, and free of undesirable particulate matter. The compositions may be sterilized by conventional sterilization techniques.

As used herein, breakpoint value refers to an active agent's concentration in the serum of a patient that produces a positive clinical response. Bacteria that are susceptible to an active agent(s) are killed at or above the breakpoint value. In the embodiments of the invention that include a combination of active agents, the breakpoint value is that for the combination.

Qualitative end points. According to CLSI standards % the MIC is defined as the lowest concentration of an antimicrobial agent that prevents visible growth in the challenge medium after a set period of incubation. By contrast, the MBEC is defined as the lowest concentration of antimicrobial agent that prevents visible growth from occurring in the recovery medium used to collect biofilm cells¹⁷. The MBEC can be determined using the protocol presented here, and is measured after the recovery medium has been incubated for a suitable period of time, the length of which depends on the growth rate of the microorganism.

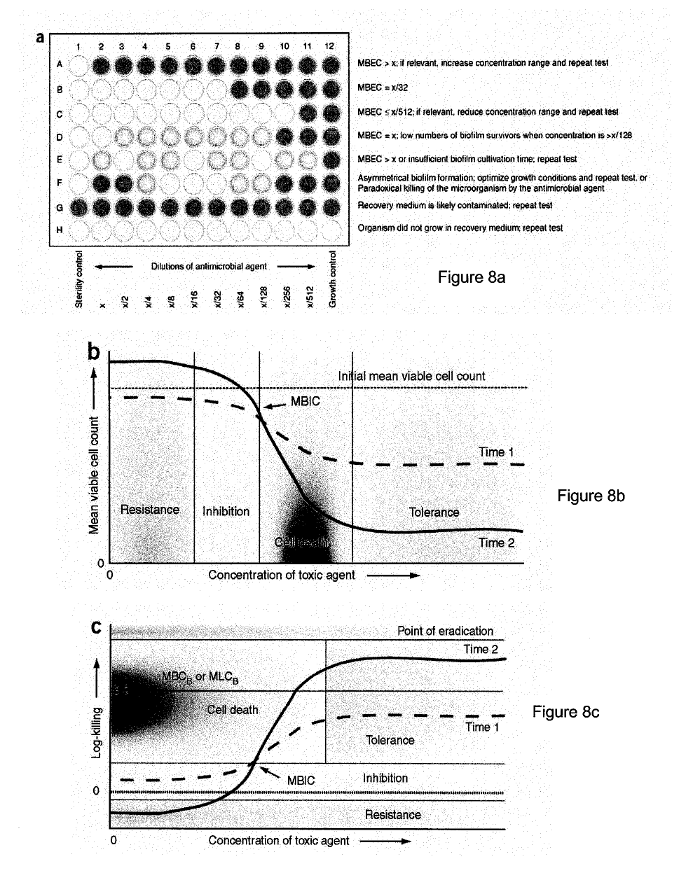

Quantitative end points. According to guidelines set by the CLSI, the minimum bactericidal concentration (MBC) is defined as the lowest concentration of an antimicrobial agent required to kill 99.9% of the starting planktonic bacterial population (or 3.0 on the log₁₀ scale). This definition can be extended to both planktonic and biofilm cells and these end points will be denoted as the MBCP and MBCB, respectively. Consistent with these guide-lines and the definitions provided by Fothergill³⁷ for the testing of planktonic yeast cells, fungal minimum lethal concentrations for planktonic cells (MLCP) and biofilms (MLCB) can be defined as the lowest concentration of an antimicrobial agent required to kill 99.5% of the starting fungal population (which also approximates 3.0 on the log₁₀ scale). The technique presented here allows the MBCB or MLCB to be calculated. Another measure of biofilm susceptibility that has been proposed is the minimum biofilm inhibitory concentration (MBIC)^{38,39}. Here, the MBIC is defined as the lowest concentration of an antimicrobial at which there is no time-dependent increase in biofilm MVCC when an early exposure time is compared with a later exposure time. The MBIC thus corresponds to the intersecting point of two concentration-dependent killing curves, and therefore, it is possible to distinguish between biofilm resistance and tolerance on the basis of VCC (FIG. 8).

Susceptibility may be determined by comparing the breakpoint susceptibility of an organism with either the attainable blood or urine level of the antimicrobial agent. The following table lists the interpretive criteria as indicated in the CLSI document M100-S9 or M100-S16.

Interpretive Breakpoints

| Antimicrobial Agent | Susceptible | Intermediate | Resistant |
|---|---|---|---|
| Amikacin | <16 | 32 | >64 |
| Aztreonam | <8 | 16 | >32 |
| Cefepime | <8 | 16 | >_ 32 |
| Ceftazidime | <8 | 16 | >32 |
| Chloramphenicol | <8 | 16 | >32 |
| Ciprofloxacin | <1 | 2 | >4 |
| Colistin | <2 | — | >4 |
| Gentamicin | <4 | 8 | >16 |
| Meropenem | <4 | 8 | >_ 16 |
| Piperacillin/tazobactam | <16/4 | 32/4-64/4 | >128/4 |
| Trimethoprim/sulfamethoxazole | <2/38 | — | >4/76 |
| Tobramycin | <4 | 8 | >16 |
| Amikacin/aztreonam | <16/8 | 32/16 | >64/32 |
| Amikacin/cefepime | <16/8 | 32/16 | >64/32 |
| Amikacin/ceftazidime | <16/8 | 32/16 | >64/32 |
| Amikacin/ciprofloxacin | <16/1 | 32/2 | >64/4 |
| Amikacin/colistin | <16/2 | — | >64/4 |
| Amikacin/meropenem | <16/4 | 32/8 | >64/16 |
| Amikacin/piperacillin/tazobactam | <16/16/4 | 32/32/4-32/64/4 | >64/128/4 |
| Amikacin/trimethoprim/sulfamethoxazole | <16/2/38 | — | >64/4/76 |
| Chloramphenicol/ceftazidime | <8/8 | 16/16 | >32/32 |
| Chloramphenicol/meropenem | <8/4 | 16/8 | >32/16 |
| Ciprofloxacin/aztreonam | <1/8 | 2/16 | >4/32 |
| Ciprofloxacin/colistin | <1/2 | — | >4/4 |
| Ciprofloxacin/meropenem | <1/4 | 2/8 | >4/16 |
| Ciprofloxacin/piperacillin/tazobactam | <1/16/4 | 2/32/4-2/64/4 | >4/128/4 |
| Ciprofloxacin/trimethoprim/sulfamethoxazole | <1/2/38 | — | >4/4/76 |
| Gentamicin/aztreonam | <4/8 | 8/16 | >16/32 |
| Gentamicin/cefepime | <4/8 | 8/16 | >16/32 |
| Gentamicin/ceftazidime | <4/8 | 8/16 | >16/32 |
| Gentamicin/ciprofloxacin | <4/1 | 8/2 | >16/4 |
| Gentamicin/colistin | <4/2 | — | >16/4 |
| Gentamicin/meropenem | <4/4 | 8/8 | >16/16 |
| Gentamicin/piperacillin/tazobactam | <4/16/4 | 8/32/4-8/64/4 | >16/128/4 |
| Gentamicin/trimethoprim/sulfamethoxazole | <4/2/38 | — | >16/4/76 |
| Tobramycin/aztreonam | <4/16 | 8/32 | >16/64 |
| Tobramycin/cefepime | <4/8 | 8/16 | >16/32 |
| Tobramycin/ceftazidime | <4/8 | 8/16 | >16/32 |
| Tobramycin/ciprofloxacin | <4/1 | 8/2 | >16/4 |
| Tobramycin/colistin | <4/2 | — | >16/4 |
| Tobramycin/meropenem | <4/4 | 8/8 | >16/16 |
| Tobramycin/piperacillin/tazobactam | <4/16/4 | 8/32/4-8/64/4 | >16/128/4 |
| Tobramycin/trimethoprim/sulfamethoxazole | <4/2/38 | — | >16/4/76 |
| Trimethoprim/sulfamethoxazole/aztreonam | <2/38/16 | — | >4/76/64 |
| Trimethoprim/sulfamethoxazole/ceftazidime | <2/38/8 | — | >4/76/32 |
| Trimethoprim/sulfamethoxazole/meropenem | <2/38/4 | — | >_4/76/16 |
| Trimethoprim/sulfamethoxazole/piperacillin/ | <2/38/16/4 | — | >4/76/128/4 |

Terminology. Resistance is defined as the ability of a microorganism to continue growing in the presence of an antimicrobial agent. The MIC and MBIC are measures of planktonic cell and biofilm resistance, respectively. By contrast, tolerance is defined as the ability of a microorganism to survive, but not grow, in the presence of an antimicrobial agent. The MBEC, MBC and MLC are measures of tolerance. FIG. 8 provides an example of how to interpret these measurements.

McFarland standards Originally described in 1907, McFarland standards are used as a reference to adjust the turbidity of bacteria in suspension⁵³. This calibration is based on OD and is widely used in susceptibility testing to ensure that consistent starting numbers of microorganisms are used from one experiment to the next. McFarland OD standards prepared from latex beads can be purchased from one of several suppliers, or alternatively, these can be prepared in the laboratory. To do this, prepare a 1.0% (wt/vol) solution of anhydrous barium chloride (BaCl₂, 0.048 mol 1⁻¹) and a 1.0% (vol/vol) solution of sulfuric acid (H₂SO₄, 0.18 mol 1⁻¹). Alternatively a 1.175% solution of barium chloride dihydrate (BaCl₂.2H₂O) could be used instead of the anhydrous BaCl₂ salt. One skilled in the art may determine the appropriate volumes of these solutions that may be mixed to obtain the desired McFarland standard reference. Prepare standards in clear, screw-capped glass tubes that are of the same diameter as those used for preparing the bacterial suspension for inoculation. Seal the tubes tightly with Parafilm to prevent evaporation. Use a vortex mixer to suspend the barium sulfate (BaSO₄) precipitates in the McFarland standards before each use. Note that commercial standards containing latex beads should not be vortexed and instead, these can be mixed by inverting the tubes several times. Standards can be stored in the dark at room temperature (20-25° C.) for up to 6 months, after which they should be discarded. H₂SO₄ is toxic and corrosive. Always wear gloves and safety clothing when handling this acid. The growth phase of inoculating microbes is of paramount importance in susceptibility testing. Bacteria have an extended and variable lag phase after stationary-phase growth, which could impact biofilm cultivation in a different way on a day-to-day basis. The McFarland standard strategy for inoculation used in the core protocol calibrates starting cell number based on OD of the colonies picked from a fresh agar plate or broth culture and circumvents this problem. This strategy is consistent with CLSI protocols for standardized MIC testing.

When testing for asymmetrical biofilm formation across the peg lid, one-way ANOVA may be used to compare the log₁₀-transformed, dilution factor-corrected plate counts for 48 of the pegs in the device (wells 1-6 from rows A to H of the peg lid. The VCCs are grouped by row of the peg lid, and one-way ANOVA is tested at the 5% level of confidence using a statistical software package such as MiniTab 15 (Minitab, State College, Pa., USA). If P≦0.05, then the null hypothesis that the mean biofilm cell density in each row of the peg lid is equivalent is rejected. This indicates that the growth conditions might need to be adjusted or that the equipment, such as the rocking table or the orbital shaker, might need to be calibrated. If P >0.05, then there is no significant difference between cell density in the different regions of the device and this indicates that the growth conditions are suitable for biofilm susceptibility testing.

Most bacterial and fungal media can be purchased from suppliers or they can be prepared from ingredients according to existing protocols in the literature. If prepared from powdered forms, dissolve media in ddH₂O and adjust the pH as required. Autoclave (121° C. for 30 min, 23 p.s.i.) or filter sterilize all media before use. Dry the surface of agar media leaving Petri dishes to sit at room temperature for 2 d; alternatively, after agar medium has set, dry the surface of the agar in an incubator or a biological safety cabinet for 30 min, with the lid of the Petri dish kept ajar. It is essential that the agar surface be sufficiently dry to obtain accurate counts by a spot plating technique. Once prepared, most microbiological media can be stored at 4° C. for up to several months. Agar plates should be stored bottom-up to prevent moisture from accumulating on the agar surface.

The solution for rinsing biofilms and for making serial dilutions of recovered biofilm cells is an important choice. Salinity of the rinse solution can affect cell viability and thus it may be necessary to use PBS for some microorganisms and ddH₂O for others. Certain buffers might also affect susceptibility testing and, hence, compatibility of the rinse solution with the test agents must be carefully considered. For instance, biofilms tested against CuSO₄ should not be rinsed with PBS, as phosphates may be carried over to the exposure step and copper phosphates, which are biologically less available forms of Cu, can readily form even in those media specifically formulated for metal susceptibility testing⁵⁰. Autoclave the rinse solution to sterilize it. A sterile rinse solution may be stored at room temperature for up to 6 months.

### EXAMPLES

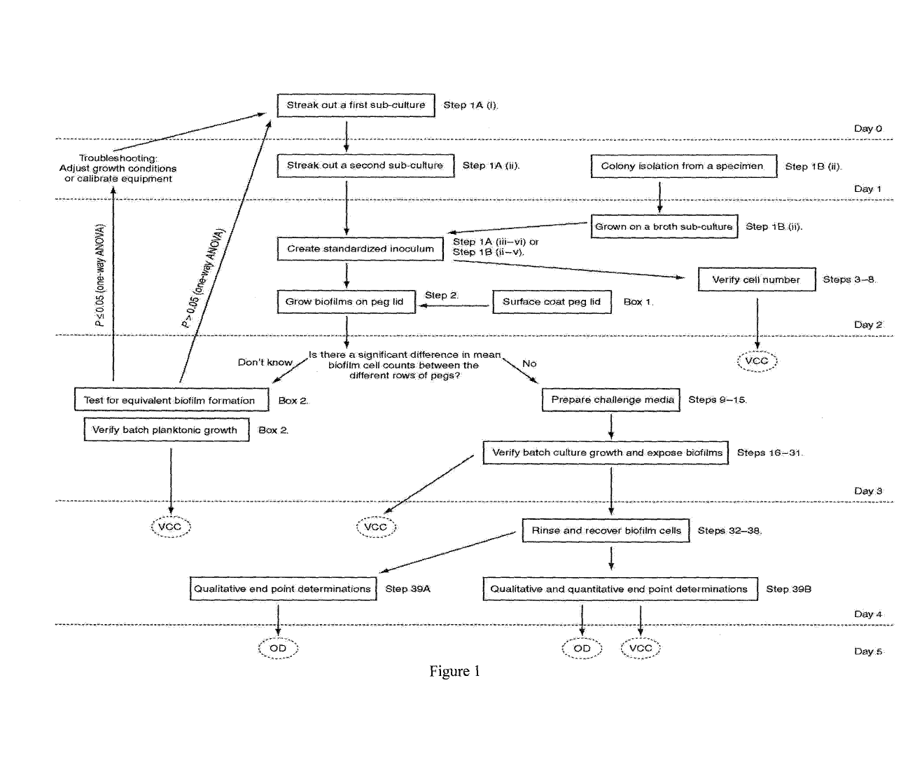

The following protocol is used for the examples, except where noted. An example of a protocol of the present invention is shown in FIG. 1.

Antibiotic and other antimicrobial stock solutions should be prepared in advance at 5× the highest concentration to be used in the challenge plate. For example, de-ionized water or an appropriate solvent is used to prepare stock solutions of antibiotics at 120 μg ml⁻¹ of active agent. Consult Clinical Laboratory Standards Institute (CLSI) document M100-S8 for details of which solvents and diluents to use.

Stock solutions of antibiotics and other antimicrobial agents The solubility of antimicrobial agents can vary considerably and thus chemistry dictates the choice of solvent. Many drugs and antimicrobial agents are water soluble, but some will require solvents other than water (consult the manufacturer's instructions or The Merck Index⁵¹). Prepare stock agents at 5× concentrations, the highest concentration to be tested against biofilms. Split stock solutions of antibiotics into aliquots and store at −70° C. Most antibiotics are stable at this temperature for at least 6 months (see Andrews⁵² for information on specific antibiotics). Solutions of metal ions and industrial biocides can be stored at room temperature in air-tight containers for at least 1 month, although this might vary by compound (consult the manufacturers' guidelines). Many stock antibiotic and metal ion solutions can be filter sterilized using a 0.22 μm membrane syringe filter; however, it is important to ascertain that the compound will not adsorb to the membrane filter (consult the manufacturer's guidelines). In some cases, filter sterilization is neither required nor recommended, such as in the cases of most disinfectants, biocides and antimicrobial peptides'. Instead, prepare these agents using semi-sterile technique: autoclave the utensils used to handle the compounds and filter sterilize the solvent before dissolving the antimicrobial agent in it. Many antibiotics are standardized by biological assays performed by the manufacturer. The specific activity of the antibiotic (expressed in U mg⁻¹) must be used to correct for the amount of antibiotic used to make up the stock solution. This standardization ensures day-to-day reproducibility of susceptibility data.

It is sometimes appropriate to employ a neutralizing agent for determining minimum bactericidal and fungicidal concentrations. These agents reduce toxicity from the carry-over of biologically active compounds from challenge to recovery media. For example, β-lactamase may be used to neutralize penicillin, or L-cysteine may be used to neutralize Hg²⁺ or some other heavy metal cations. The following experiments use a universal neutralizer in biocide susceptibility assays comprising 1.0 g L-histidine, 1.0 g L-cysteine, and 2.0 g reduced glutathione. Make up to 20 ml in double distilled water. Pass through a syringe with a 0.20 to 0.22 μm filter to sterilize. This solution may be stored at −20° C. Make up 1 liter of the appropriate growth medium (e.g., cation adjusted MHB). Supplement this medium with 20.0 g per liter of saponin and 10.0 g per liter of Tween-80. Adjust with dilute NaOH to the correct pH (7.0±0.2 at 20° C.). Add 500 μl of the universal neutralizer to each 20 ml of the surfactant supplemented growth medium used for recovery plates.

An overview of this experimental protocol is provided in FIG. 1. The number of days required to complete this protocol is dependent on the growth rate of the microorganism being examined. The protocol has been divided into 6 sequential steps, each of which is detailed below.

This protocol has been developed for use with Nunc Brand, flat bottom, 96-well microtiter plates. These microplates have a maximum volume of 300 μl per well. The medium and buffer volumes listed here may need to be adjusted for different brands of microtiter plates.

### Example 1

### Step 1—Growing Sub-Cultures of the Desired Microorganism.

1. If using a cryogenic stock (at −70° C.), streak out a first sub-culture of the desired bacterial or fungal strain on an appropriate agar plate. Incubate at the optimum growth temperature of the microorganism for an appropriate period of time. For most bacterial strains, the first sub-culture may be wrapped with Parafilm™ and stored at 4° C. for up to 14 days.
2. Check the first sub-culture for purity (i.e. only a single colony morphology should be present on the plate).
3. From the first sub-culture or from a clinical isolate, streak out a second sub-culture on an appropriate agar plate. Incubate at the optimum growth temperature of the microorganism for an appropriate period of time. The second sub-culture should be used within 24 h starting from the time it was first removed from incubation.
4. Verify the purity of the second sub-culture.

It is not recommended to grow subcultures on media containing selective agents. Antibiotics and other antimicrobials may trigger an adaptive stress response in bacteria and/or may increase the accumulation of mutants in the population. This may result in an aberrant susceptibility determination.

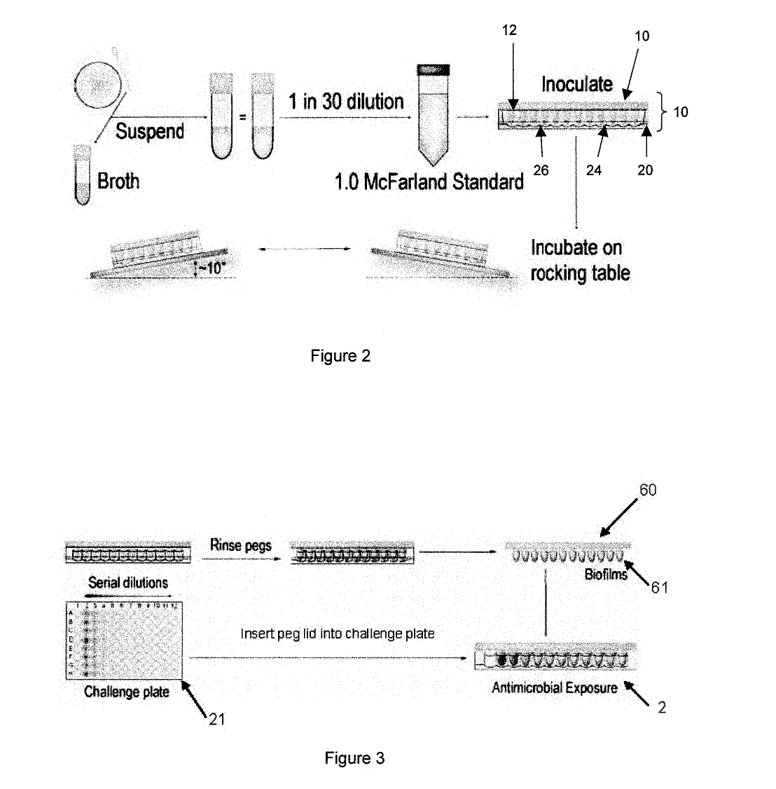

### Step 2—This Step, Inoculating the Assembly, is Illustrated in FIG. 2.

In summary, a fresh second sub-culture is used to create an inoculum that matches a 1.0 McFarland Standard. This solution is diluted 1 in 30 with growth medium. 22 ml of the 1 in 30 dilution is added to the trough of the base in an assembly of the present invention. The device is placed on a rocking table to assist the formation of biofilms on the polystyrene pegs.

It is recommended that the following steps be carried out in a biological safety cabinet (if available). However, it is possible to use aseptic technique on a bench top:
1. Open a sterile 96-well microtiter plate. For each high throughput assay, fill 4 ‘columns’ of the microtiter plate from ‘rows’ A to F with 180 μl of a physiological saline solution.
2. Put 1.5 ml (plus 1.0 ml for each additional device being inoculated at the same time) of the desired broth growth medium into a sterile glass test tube.
3. Using a sterile cotton swab, collect the bacterial colonies on the surface of the second agar sub-culture. Cover the tip of the cotton swab with a thin layer of bacteria.
4. Dip the cotton swab into the broth to suspend the bacteria. The goal is to create a suspension that matches a 1.0 McFarland standard (i.e. 3.0×10⁸ cfu ml⁻¹). Be careful not to get clumps of bacteria in the solution.
5. Repeat step 2, parts 3 and 4 as many times as required to match the optical standard.
6. Put 29 ml of the appropriate broth growth medium (e.g. TSB) into a sterile 50 ml polypropylene or glass tube. To this, add 1.0 ml of the 1.0 McFarland standard bacterial suspension. This 30 fold dilution of the 1.0 McFarland standard (i.e. 1.0×10⁷ cfu ml⁻¹) serves as the inoculum for the device.
7. Open the sterile package of the device. Pour the inoculum into a reagent reservoir. Using a sterile pipette, add 22 ml of the inoculum to the trough packaged with the device. Place the peg lid onto the trough.

The volume of inoculum used in this step has been calibrated such that the biofilm covers a surface area that is immersed, entirely, by the volume of antimicrobials used in the challenge plate set up in Step 3 (below). Using a larger volume of inoculum may lead to biofilm formation high on the peg that physically escapes exposure in this challenge step.
8. Place the device on the rocking table in a humidified incubator at the appropriate temperature. The table should be set to between 3 and 5 rocks per minute. The Inventors have found that setting the angle of the rocking table to between 9° and 16° of inclination provides biofilm growth with the appropriate cell density. This motion must be symmetrical. The target is to generate a biofilm of >10⁵ cfu/peg, usually 24 hour incubation is sufficient.
9. Serially dilute (ten-fold) a sample of the inoculum (do 3 or 4 replicates). These are controls used to verify the starting cell number in the inoculum (should contain approx. 1×10⁷ cfu/mL) and to check for contaminants in the culture.
10. Spot plate the serial 10 fold dilutions of the inoculum from 10⁻⁶ to 10⁻¹ on an appropriately labeled series of agar plates. Incubate the spot plates for an appropriate period of time and score for growth.

Sterility Controls (optional). Using alcohol flamed pliers, break off pegs A1, B1, C1 and D1 such that there will no longer be protrusions to which bacteria could adhere. These positions will serve as sterility controls for the assay.

### Example 2

### Step 3—Set Up the Antimicrobial Challenge Plate.

The following section describes how to set up a serial two-fold dilution gradient of a single antimicrobial in the challenge plate. The antimicrobial challenge plate may be set up in any manner desired with any combination of antimicrobials. It is important that the final volume in each well of the challenge plate is 200 μl in order to ensure complete submersion of the biofilm in the antimicrobial composition. Consult NCCLS document M100-S8 for details on which solvents and diluents to use.
1. Open a sterile 96-well microtiter plate in a laminar flow hood.
2. Setup a working solution of the desired antimicrobial in the appropriate growth medium. Do not dilute the antimicrobial by more than 20% (i.e., no more than 1 part stock antimicrobial solution per 4 parts of growth medium). The working solution of the antimicrobial should be made at a concentration equal to the highest concentration to be tested in the challenge plate. An example of how the challenge plate can be prepared follows.
3. Add 200 gl of growth medium to ‘column’ 1 and ‘column’ 12 of the challenge plate. These will serve as sterility and growth controls, respectively.
4. Add 100 μl of growth medium to ‘columns’ 3 to 11 of the microtiter plate.
5. Add 200 μl of the working solution to ‘column’ 2 of the microtiter plate.
6. Add 100 μl of the working solution to ‘column’ 3 and ‘column’ 4 of the microtiter plate.
7. Using the multi-channel micropipette, mix the contents of ‘column’ 4 by pipetting up and down. After mixing, transfer 100 μl from the wells in ‘column’ 4 to the corresponding wells in ‘column’ 5.
8. Mix and transfer 100 μl from ‘column’ 5 to ‘column’ 6. Serially repeat this mix and transfer process down the length of the microtiter plate until reaching ‘column’ 11.
9. Mix the contents of column 11 up and down. Extract 100 μl from each well in ‘column’ 11 and discard.
10. Add 100 μl of growth media to the wells in ‘columns’ 4 through 11.
11. Replace the lid on the challenge plate. Gently tap the plate to facilitate mixing of biocide/antibiotic and media.

Alternatively, designate the antibiotics to be tested in the assay and assign them to rows A through H. This example of a plate set-up will allow 8 different antibiotics at 10 concentrations to be tested. These concentrations can be adjusted accordingly to suit the needs of the study, or of the biofilm(s) being tested.

### Step 4—Expose the Biofilms.

This step, exposing the biofilm to one or more anti-microbials, is illustrated in FIG. 3. In summary, the assembly prepared above is removed from the gyrorotary shaker and the biofilms are rinsed in a physiological saline solution. The rinsed biofilms are then immersed in the antimicrobials of the challenge plate and incubated for the desired exposure time.
1. Set up a sterile microtiter plate with 200 μl of physiological saline solution in every well. This plate will be used to rinse the pegs to remove loosely adherent planktonic cells from the biofilm (this is termed a ‘rinse plate’).
2. This step will be used to determine biofilm growth on four sample pegs and from four wells of the planktonic cultures. Setup a sterile microtiter plate with 200 μl of physiological saline solution in 4 ‘columns’ of row A for each device inoculated (i.e., 1 microtiter plate is required for every 3 devices). Fill rows B to F with 180 μl of physiological saline solution. In a second microtiter plate, fill 4 ‘columns’ from rows A to H with 180 μl of physiological saline solution for each device inoculated. The first microtiter plate will be used to do serial dilutions of biofilm cultures, the second will be used to check the growth of planktonic cells in the wells of the microtiter plate that contained the inoculum.
3. Following the desired period of incubation, remove the high throughput assembly from the rocking table and into the laminar flow hood. Remove the peg lid from the trough and submerse the pegs in the wells of the rinse plate. Let the rinse plate sit for 1 to 2 minutes while performing step 4 below.
4. Use a micropipette to transfer 20 μl of the planktonic culture (in the corrugated trough of the device) into the 180 μl of saline in row ‘A’ of the latter plate set up in step 2 (immediately above). Repeat this three more times for a total of 4×20 μl aliquots.
5. Discard the planktonic culture into the appropriate biohazard waste.
6. In the laminar flow hood, dip a pair of pliers into 95% ethanol. Flame the pliers using the ethanol lamp in the flow hood. Be cautious when using the ethanol lamp. Do not light the ethanol lamp and do not flame the pliers before your hands have dried following disinfection using 70% ethanol.
7. Using the flamed pliers, break off pegs A1, C1, E1 and G1 from the lid of the assembly and immerse them in the 200 μl of saline in row A (and each in a different ‘column’) of the first plate setup in step 2.
8. Using the flamed pliers, break off pegs B1, D1, F1 and H1 and discard.
8a. Biofilm inoculum check (optional): using flamed pliers remove pegs E1, F1, G1, and H1, placing each in 200 μL saline in a dilution plate. Sonicate the sample pegs E1-H1 for 5 minutes on high to dislodge the biofilm bacteria then serially dilute to 10-7 and spot plate on TSA (or appropriate media) and incubate overnight to determine cfu/peg.
9. Insert the peg lid of the assembly into the challenge plate. Place the challenge plate in the appropriate incubator for the desired exposure time. Incubations may be carried out at alternative temperatures, taking into consideration extended times for MIC determinations.
10. Place the microtiter plate containing the sample pegs in the tray of the ultrasonic cleaner (the sonicator). Sonicate on the setting ‘high’ for 5 to 30 minutes (the time required depends on the microorganism being assayed). The vibrations created in the water by the sonicator transfer first through the water, then through the steel insert tray, and finally to the device to use vibrations to disrupt biofilms from the surface of the 96 pegs into the saline.
11. Serially dilute 20 μl aliquots of the planktonic cultures (from step 4) in the wells of the corresponding microtiter plate. Once sonication is complete, repeat this serial dilution process with the biofilm cultures.
12. Spot plate the serial 10 fold dilutions of the planktonic and biofilm cultures from 10⁻⁸ to 10⁻³ and 10⁻⁵ to 10⁰ on an appropriately labeled series of agar plates. Incubate the spot plates for an appropriate period of time and score for growth.

### Step 5—Neutralize and Recover.

This step, neutralizing the anti-microbials and recovering surviving biofilm bacteria, is illustrated in FIG. 4. In summary, after exposure, biofilms are rinsed twice in physiological saline. The biofilms are then transferred to a microtiter plate containing a neutralizing agent and recovery medium. The biofilms are disrupted into this by sonication on a water table sonicator.
1. Add 200 μl of the appropriate recovery medium (e.g., containing a neutralizing agent) to each well of a brand new 96-well microtiter plate. This plate is termed the ‘recovery plate’.
2. Prepare 2 rinse plates for every assembly used.
3. Remove the challenge plate from the incubator and place in the laminar flow hood (or use careful aseptic technique). Remove the peg lid and immerse the pegs in the physiological saline of a rinse plate. Cover the challenge plate with the sterile lid of the rinse plate. After approximately 1 min, transfer the peg lid from the first rinse plate into the second rinse plate. Cover the challenge plate and retain for an MIC determination if appropriate.
4. Transfer the peg lid from the second rinse plate into the recovery plate setup above. Transfer the recovery plate (containing the pegs of the device) onto the tray of the sonicator. Sonicate on high for 5 to 30 min. (depending on the thickness of the biofilm). The vibrations will disrupt biofilms from the surface of the 96 pegs into the recovery plate.
5. After sonication, remove the peg lid from the recovery plate and replace the original lid of the microtiter plate. The lid of the device may now be discarded into autoclave garbage.
6. Place the recovery plate in the incubator and incubate a minimum of 24 to 72 h, depending on the organism being examined.

### Viable Cell Counting

For viable cell counts of biofilms after treatment with an antimicrobial, transfer 100 μl of the recovery media (containing the sonicated biofilms) from the recovery plate to row A of a serial dilution plate. This plate may contain 180 μl of physiological saline solution in each well of rows B to F. Serially dilute 20 μl from row A using the multi-channel pipette. Ensure that the tips on the multi-channel pipette are changed between transfers to each row in the microtiter plate. Spot plate biofilm cultures (which have been serially diluted ten-fold) on appropriately labeled agar plates. Incubate for a minimum of 48 hours to ensure maximum recovery of the surviving microorganisms.

Following incubation, enumerate bacteria recovered on plates. Use the formulas in the following section to determine killing of the biofilm population. To calculate death and survival (log-kill), use the following formula:

log-kill=log₁₀(initial cfu/ml)−log₁₀(remaining cfu/ml after exposure)

Alternatively,

log-kill=log₁₀[1/(1−% kill (as a decimal))]

To calculate percent kill, use the following formula:

% kill=[1−(remaining cfu/ml)/(initial cfu/ml)]×100

To calculate percent survival, use the following formula:

% survival=[(remaining cfu/ml after exposure)/(initial cfu/ml)]×100

To calculate log percent survival, use the following formula:

log % survival=log₁₀(% survival)

### Microscopy

For many microscopy techniques, it may be desirable to fix the biofilms to the surface of the pegs of the assembly. The following protocols may be used to prepare biofilms for scanning electron microscopy (SEM) and confocal laser scanning microscopy (CLSM). In the standard experimental procedure above, each challenge plate has eight growth controls (before exposure). Four of these are used for growth controls. The remaining four may be used for microscopy instead of being discarded.

### Fixing Biofilms for Scanning Electron Microscopy (SEM)

### Preparing Working Solutions

Wear protective gloves in the following steps and handle these highly toxic chemicals in a fume hood.

Cacodylate buffer 0.1 M: dissolve 16 g of cacodylic acid in 1 liter of double distilled H₂O; adjust to pH 7.2.

Glutaraldehyde 2.5% in cacodylate buffer: dissolve 2 ml of 70% glutaraldehyde in 52 ml of cacodylate buffer (yields a 2.5% solution). It is also possible to use a 5% solution (2 ml of glutaraldehyde into 26 ml of cacodylate buffer).

### Standard Protocol

This fixing technique is destructive to biofilms. However, this allows an examination of the cell structure of the underlying bacteria.
1. Break pegs from the MBEC™-HTP device using a pair of flamed pliers.
2. Rinse pegs in 0.9% saline for 1 min. This disrupts loosely-adherent planktonic bacteria.
3. Fix the pegs in 2.5% glutaraldehyde in 0.1 M cacodylic acid (pH 7.2). Pegs are placed in this solution at 4° C. for 16 h.
4. Following this fixing step, wash the pegs once in 0.1 M cacodylic acid for approximately 10 min.
5. Wash the pegs once in double distilled water for approximately 10 min.
6. Dehydrate the pegs in 70% ethanol for 15 to 20 minutes.
7. Air dry for a minimum of 24 h.
8. Mount specimens and examine by SEM.

### Alternative Protocol

This fixing technique is less destructive. It is possible to observe the extracellular polymeric matrix and some (albeit dehydrated) biofilm structure.
1. Break pegs from the MBEC™-HTP device using a pair of flamed pliers.
2. Rinse pegs in 0.9% saline for 2 min. This disrupts loosely-adherent planktonic bacteria.
3. Fix the pegs in 2.5% glutaraldehyde in 0.1 M cacodylic acid (pH 7.2). Pegs are placed in this solution at 20° C. for 2 to 3 h.
4. Air dry for at least 120 h.
5. Mount specimens and examine by SEM.

### Fixing Biofilms for Confocal Scanning Laser Microscopy (CLSM)

Glutaraldehyde 5% in phosphate buffered saline: dissolve 2 ml of 70% glutaraldehyde in 26 ml of phosphate buffered saline (yields a 5% solution).

### Standard Protocol

1. Break pegs from the lid using a pair of flamed pliers.
2. Rinse pegs in 0.9% saline for 1 min. This disrupts loosely-adherent planktonic bacteria.
3. Fix the pegs in 5% glutaraldehyde in phosphate buffered saline (pH 7.2). Pegs are placed in this solution at 30° C. for 0.5 to 1 h.
4. Rinse pegs in 0.9% saline for 1 min.
5. Stain pegs with the appropriate fluorphores and examine using the confocal laser scanning microscope.

### Example 3

### Determine MBEC Values

To determine the minimum biofilm eradication concentration (MBEC) values, check for turbidity (visually) in the wells of the recovery plate. Alternatively, use a microtiter plate reader to obtain optical density measurements at 650 nm (OD₆₅₀). Clear wells (OD650<0.1) are evidence of biofilm eradication.

### Example 4

### Determine MIC Values

To determine the minimum inhibitory concentration (MIC) values, check for turbidity (visually) in the wells of the challenge plate. Alternatively, use a microtiter plate reader to obtain optical density measurements at 650 nm (OD₆₅₀). The MIC is defined as the minimum concentration of antibiotic that inhibits growth of the organism. Clear wells (OD₆₅₀<0.1) are evidence of inhibition following a suitable period of incubation.

### Example 5

Pseudomonas aeruginosa (Pa) and Staphylococcus aureus (Staph) form biofilms on tissue and implanted surfaces resulting in persistent infections that are frequently unresponsive to conventional antimicrobial therapy, believed to be due in part to biofilm-specific resistance mechanisms. The use of MIC to select antimicrobial therapeutics for biofilm infections is therefore usually not suitable. An assay of the present invention was used for evaluation of antimicrobial susceptibility of biofilm and planktonic bacteria to single and combinations of agents.

Biofilms of Pseudomonas aeruginosa (12 isolates from Cystic Fibrosis patients) were formed on the pins of a device lid of the present invention. Biofilm and Planktonic bacteria were then exposed to various antibiotic and antibiotic combinations for 24 hours (Table 1). The assay provides qualitative sensitivity of each isolate as a biofilm and planktonic organism to antimicrobial agents alone or in combination.

Results:

TABLE 1
Pseudomonas resistance to individual
antibiotics and antibiotic combinations

| Antibiotic | Planktonic | Biofilm | Antibiotic | Planktonic | Biofilm |
|---|---|---|---|---|---|
| GM/AZTR | 1 | 12 | CLO/TMS | 0 | 3 |
| GM/CFTZ | 3 | 12 | CFTZ/AZTR | 11 | 12 |
| TB/AZTR | 1 | 12 | CIPRO/AK | 5 | 12 |
| TB/CFTZ | 3 | 12 | CIPRO/AZTR | 0 | 3 |
| P + T/TB | 1 | 12 | P + T | 4 | 12 |
| P + T/GM | 1 | 12 | CLO | 2 | 12 |
| AK/AZTR | 2 | 12 | AZTR | 0 | 9 |
| AK/P + T | 2 | 12 | CIPRO | 4 | 12 |
| TB/CIPRO | 3 | 12 | GM | 8 | 12 |
| TB/IMP | 1 | 12 | AK | 8 | 11 |
| GM/IMP | 8 | 12 | TB | 3 | 12 |
| CLO/RIF | 8 | 12 | TMS | 1 | 6 |
| AK/CFTZ | 2 | 12 | CFTZ | 3 | 12 |
| AK/IMP | 4 | 11 | IMP | 12 | 12 |

Conclusions: Pseudomonas strains were sensitive to multiple antibiotics as planktonic forms but significantly more resistant as a biofilm. Certain antibiotics were more effective as combinations than as individual agents.

### Example 6

In the following example, a device of the present invention was used. This medical device was specifically developed for testing planktonic and biofilm susceptibility at serum breakpoint levels of clinical isolates putatively containing Pseudomonas aeruginosa.

A device of the present invention was used for testing planktonic and biofilm susceptibility of clinical isolates of the opportunistic bacterial pathogen Pseudomonas aeruginosa at serum breakpoint levels. Qualitative antimicrobial agent susceptibility information was provided simultaneously for 12 single antibiotics and 35 combination antibiotics tested against planktonic and biofilm (sessile) growth forms of the organism.

96 equivalent biofilms of the clinical isolate were first formed on the high throughput (HTP) Assay under flow conditions. In a 96 well platform (Nunc™ brand) a range of antimicrobial agents alone and in combination are diluted in cation adjusted Mueller-Hinton Broth (CAMHB) at categorical breakpoint concentrations, as determined by the Clinical and Laboratory Standards Institute (CLSI) and British Society for Antimicrobial Chemotherapy. Wells were inoculated with planktonic and biofilm P. aeruginosa using the 95 peg inoculation device. Panels were incubated at 35° C. for 16-24 hours. Planktonic susceptibility and resistance was then determined by measuring growth in the wells in the presence of the antimicrobial agents.

The pegged lid containing the biofilm bacteria that have been exposed to antimicrobial agents was then placed in a recovery panel containing only CAMHB in its wells. Biofilm susceptibility and resistance was determined by measuring growth after incubation for an additional 16-24 hours at 35° C.

The following data was obtained from 14 hospitalized anonymous CF patients:5 of the 14 patients had antibiotics changed directly as a result of the MBEC data.

Of the 14 patients only 5 have sufficient data on clinical isolates in their notes to assess whether the results caused a reduction in the number and quantity of P. aeruginosa isolated. Of the 5 patients 2 demonstrated a reduction in the quantity of P. aeruginosa isolated and for 3 patients no change was observed.

8 of the 14 patients observed changes in their lung function and spirometry after susceptibility testing.

TABLE 2
Changes which occurred as a result of knowledge of the results obtained from
bioFILM PA ™ Susceptibility Kit in 14 Cystic Fibrosis (CF) patients at the CF Clinic of
the University of Alberta Hospitals, Edmonton.

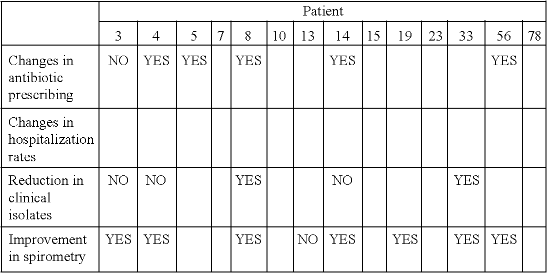

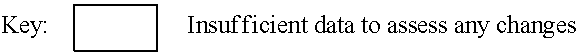

### Example 7

Refer to the table below for a summary of patient spirometry data. The spirometry data analysed in this study was pre bronchodilation forced vital capacity (pre FVC) (the maximal expiration to residual volume), and pre bronchodilation forced expirational volume in 1 second (pre FEV1).

Of the 14 patients there is sufficient spirometry data for 8 patients to assess whether biofilm susceptibility results had an impact on lung function.

7 of the 8 patients demonstrated an improvement in pre FVC of between 103 and 145% up to 134 days after susceptibility testing was performed. 6 of the 8 patients demonstrated an improvement in pre FEV1 of between 107 and 353% up to 134 days after susceptibility testing was performed and one patient demonstrated no change.

1 of the 8 patients demonstrated a reduction in lung function in the six months (189 days) proceeding biofilm susceptibility testing: pre FVC decreased from 80 to 73 (9%) and pre FEV1 decreased from 62 to 52 (8%).

TABLE 3
Changes in spirometry data as a result of knowledge of the results obtained
from biofilm susceptibility testing in 14 Cystic Fibrosis (CF) patients.

| | | Patient | | | | | | | |
|---|---|---|---|---|---|---|---|---|---|
| | | 3 | 4 | 8 | 13 | 14 | 19 | 33 | 56 |
| Pre FVC (forced | Pre biofilm | 40 | 42 | 64 | 80 | 43 | 88 | 117 | 76.7 |
| vital capacity) | susceptibility Kit | | | | | | | | |
| | results | | | | | | | | |
| | Post biofilm | 48 | 61 | 74 | 73 | 66 | 106 | 120 | 88 |
| | susceptibility Kit | | | | | | | | |
| | results | | | | | | | | |
| Pre FEV1 | Pre biofilm | 30 | 22 | 63 | 62 | 18 | 63 | 86 | 65.4 |
| (forced | susceptibility Kit | | | | | | | | |
| expirational | results | | | | | | | | |
| volume in 1 | Post biofilm | 32 | 63 | 76 | 52 | 69 | 72 | 86 | 75 |
| second) | susceptibility Kit | | | | | | | | |
| | results | | | | | | | | |
| Percentage | Pre FVC | 120 | 145 | 116 | −9 | 153 | 120 | 103 | 115 |
| change (%) | Pre FEV1 | 107 | 286 | 121 | −5 | 353 | 114 | 0 | 115 |

| | | | | | | | | |
|---|---|---|---|---|---|---|---|---|
| Time elapsed between biofilm | 62 | 122 | 76 | 189 | 85 | 134 | 91 | 37 |
| susceptibility Kit testing and | | | | | | | | |
| spirometry data (days) | | | | | | | | |

### Example 8

The CF clinic at the University Hospital has tested over 100 isolates from patients ranging from 9 to 15 years of age with a device and methods shown in the above examples. A biofilm susceptibility test was order by the doctor, and based on the test results, therapy was changed to a new combination of antibiotics.

PATIENT 1: After two weeks of new antibiotic treatment the patient improved; three of the bacterial strains were eradicated; lung function had improved by 33% from the lowest post operative measurement; and the patient was discharged from hospital.

PATIENT 2: A biofilm susceptibility test was order by the doctor, and based on the test results, therapy was changed to a new combination of antibiotics before transplant surgery.

Within two days of admission donor lungs became available and the patient underwent a successful double lung transplantation; the patient was kept on the pre-operative intravenous drug regimen during the recovery from surgery; in a period of over two years the patient has not required antibiotics for lung infection. Normally patients receiving transplanted lungs see a reoccurrence of symptoms within 6-8 months. The transplant surgeons credit this to the antibiotics received in the peri-operative period.

PATIENT 3: Patient was receiving antibiotics prior to transplantation based on traditional susceptibility testing, but the transplant team were reluctant to proceed based on the patient's poor condition. A biofilm susceptibility test was order by the doctor, and based on the test results, therapy was changed to a new combination of antibiotics. The infection responded to treatment and the transplant was performed successfully; since that time (over 2 years), the patient has had only one recurrence of a lung infection and was treated as an out-patient.

PATIENT 4: Patient was receiving home intravenous antibiotics for a Pseudomonas aeruginosa lung infection. The antibiotics had been successfully used one year earlier. A biofilm susceptibility test was order by the doctor. When the test results returned, an antibiotic not often used in CF lung infections was identified and added to the treatment. The patient has not had a recurrence of symptoms and has not required antibiotics in one year.

### Example 9

Escherichia coli strain ESBL 300-1 was susceptibility tested following the susceptibility testing protocols described in the previous Examples. It was found that E. coli were resistant to more antibiotics as biofilms than as planktonic. Many of the antibiotics that could be selected for treatment were those that would not be selected empirically or on the basis of the MIC test results.

### Example 10

Burkholderia cepacia strain ATCC 17616 was susceptibility tested following the susceptibility testing protocol described in the previous Examples. It was found that B. cepacia were resistant to most antibiotics as biofilms while many antibiotics were effective against planktonic forms. Many of the antibiotics that could be selected for treatment were those that would not be selected empirically or on the basis of the MIC test results.

### Example 11

### Coating the Surface of Pegs with Agents that Promote Microbial Adhesion•Timing ˜2 H On Day 1

Not all microorganisms can stick to polystyrene and initiate biofilm formation. A way to circumvent this problem is to coat the plastic surface with a compound that instigates microbial attachment. This is functionally analogous to cell culture treatment of plastics used in tissue culture of Eukaryotic cell lines. Such an approach has been used, e.g., to cultivate biofilms of Candida tropicalis in the Calgary Biofilm Device (CBD)^{20,21,42}. Pegs lacking pretreatment with a sterile 1.0% L-lysine (or 5.0% BSA) are unevenly colonized by as few as 10-100 yeast cells per peg In contrast, coated pegs have robust biofilms containing >10⁴ cells, many of which will differentiate into hyphal cells during 48 h growth in a buffered RPMI-1640-based nutrient medium²¹ (Example 28 for culture conditions). Pegs may be coated with various different agents that promote adhesion; one is not restricted to the example of the water-soluble amino acid or protein presented here. If desired, it is possible to prepare a mock treatment (i.e., solvent with no added agent) to assess the effect of a surface coating on biofilm growth or antimicrobial susceptibility.

Solutions of L-lysine or BSA may be prepared in double distilled water and are filter sterilized. These solutions may be stored at room temperature (25° C.) for several months. Coated peg lids are typically used the same day that they are prepared.

1. In a biological safety cabinet, open sterile packages containing one reagent reservoir and one 96-well microtiter plate. Pipette 20 ml of sterile 1.0% L-lysine (or 5.0% BSA) into the reagent reservoir.

2. Using a multichannel pipette, transfer 200 μl of 1.0% L-lysine (or 5.0% BSA) solution into each well of the microtiter plate.

3. Remove the sterile peg lid from its package and insert the lid into the microtiter plate containing the coating solution.

4. Incubate for 1 h at room temperature (25° C.).

5. Remove the peg lid from the microtiter plate and place the lid upside down in a biological safety cabinet for 30 min to air dry.

6. Use the coated peg lid in the protocol for biofilm cultivation and susceptibility testing.

### Example 12

Controls for microbial growth and biofilm formation. There are three sets of controls that should be carried out to evaluate microbial growth in the peg lid biofilm reactor (FIG. 1). First, the number of cells in the inoculum should be verified by VCC (FIG. 1, Steps 3-8). This ensures that a standard number of cells are used to initiate biofilm growth in every device. Second, and after biofilm cultivation, the number of cells growing in the planktonic inoculum as well as in the peg biofilms should be determined (FIG. 1, Steps 16-31). This verifies that the microbes can reproduce in the growth medium, provides a starting biofilm cell density that can be used for log-killing calculations and makes certain that a consistent number of microbes are present on pegs from lids in one batch to those in the next. In addition, if a viable contaminant is present either in the inoculum or on the peg lid, this will be identified by contaminating colonies present on the agar plates used for growth controls. Finally, it should be ascertained whether the growth of biofilms is nonequivalent in different regions of the device (FIG. 12 and Example 13). This last control only needs to be performed once per test isolate and ensures that the equipment and device set up do not generate asymmetry in biofilm growth within the reactor. Typically, this is assessed by determining biofilm cell viability counts for half of the pegs in the device, which are grouped by row and compared using a statistical test, such as one-way analysis of variance (ANOVA). We recommend that the experimenter carry out this last control before commencing high-throughput biofilm susceptibility testing.

### Example 13

### Test For Nonequivalent Biofilm Formation•Timing ˜90 min per Isolate on Day 2

This process only needs to be carried out once per test strain to show that, under the growth conditions used to cultivate the microbes, there is no asymmetry in biofilm growth in different regions of the peg lid biofilm reactor.
1. In a biological safety cabinet, open sterile packages containing two 96-well microtiter plates and two reagent reservoirs. Pipette 25 ml of rinse solution into one reservoir and 25 ml of recovery medium into the other.
2. Using a multichannel pipette, transfer 200 μl of rinse solution into each well of the first microtiter plate and 200 μl of recovery medium into each well of the second plate.
3. Rinse the biofilms formed in Step 6 by submersing the peg lid into the wells of the microtiter plate containing the rinse solution. Let them stand for 1 min.
4. Transfer the peg lid into the microtiter plate containing the recovery medium. Retain the sterile lid of the microtiter plate so that it can be used in Step 6. Place the microtiter plate containing the peg lid into the tray of the ultrasonic cleaner (the sonicator). Disrupt the biofilms by sonicating for 10 min.
5. While the cells are being disrupted into the recovery medium, return to the biological safety cabinet and open sterile packages containing five microtiter plates. Set up the first four plates to facilitate serial dilution of the recovery medium. To do this, use the multichannel pipette to add 180 μl of rinse solution into each well of rows B to H of these four microtiter plates. Set up the fifth plate to facilitate serial dilution of the planktonic inoculum. To do this, transfer 180 μl of rinse solution into each well of columns 1-4 in this last microtiter plate.
6. When sonication is complete, retrieve the peg lid and recovery medium and return it to the biological safety cabinet. Remove and discard the peg lid in appropriate biohazardous waste container. Cover the microtiter plate containing the recovery medium with the sterile lid retained in Step 4.
7. Using a multichannel pipette, transfer 50 μl of recovery medium from wells A1-A6 into wells A1-A6 of one of the microtiter plates set up for serial dilutions in Step 5. Next, transfer 50 μl of recovery medium from wells B1-B6 into wells A7-A12 of the same microtiter plate. Repeat this transfer process for each pair of wells C1-C6 and D1-D6, E1-E6 and F1-F6, G1-G6 and H1-H6, each time arranging the 50 μl aliquots into the first 12 wells of the remaining microtiter plates set up in Step 5.
8. Using a multichannel pipette, transfer a 20 μl aliquot of the now turbid inoculum into each of the wells A1, B1, C1 and D1 of the fifth microtiter plate prepared in Step 5. If biofilms were grown in a trough format device, these four aliquots are taken from the fluid sitting in the bottom of the trough; if biofilms were grown in microtiter plates, these four aliquots can be taken from any four different wells.
9. Discard the planktonic inoculum into an appropriate biohazardous waste receptacle.
10. Use a multichannel micropipette to serially dilute 20 μl aliquots of the recovery medium and the planktonic cell culture in the sterile rinse solution in the corresponding microtiter plates. Use the same technique described in Step 6 of the protocol.
11. Using a multichannel pipette, transfer 10 μl aliquots from every well of rows F to A of the microtiter plates onto appropriately labeled agar growth medium. Use the same technique described in Step 7 of the protocol. Incubate this spot plate using the optimum growth conditions of the test organism.
12. Score the spot plates by colony counting using the same approach described in Step 8. Determine the viable cell counts for the batch planktonic culture and each peg biofilm using equation (1). Group the biofilm viable cell counts by row and compare them using one-way

ANOVA (analysis of variance). Stop the protocol here and assess the reaction set up for the anticipated planktonic growth and for nonequivalent biofilm formation.

### Example 14

### Evaluation of Cell Viability Data

Mathematical analysis. A set of statistical calculations³⁶ may be carried out to determine the number of cells in the biofilm population, and these measurements can be expressed in CFU per peg. The sample VCC, the sample mean VCC (MVCC) and sample standard deviation (SD) can be determined from the dilution factor (DF)-corrected, logᵢₐ-transformed plate counts using the following equations:

$$\begin{matrix}VCC = {log}_{10} ( platecount \times DF ) & ( 1 ) \\ MVCC = \sum \frac{VCC}{n} = \sum \frac{( {Log}_{10} ( platecount \times DF )}{n} & ( 2 ) \\ SD = \sum \frac{( VCC - {MVCC}^{2} )}{n} & ( 3 )\end{matrix}$$

where n is the number of measurements. Log-transformation is required to normalize these population data, and this normal distribution is an assumption of the statistical tests used to analyze these data. Note that as a matter of convention, a plate count of zero will result in a value of 1, as log₁₀ (0)=1. This approach is adopted as it is not mathematically possible to plot a zero value on a logarithmic scale.
Next, the sample log-kill (LK) and sample mean log-kill (MLK) for biofilm populations can be calculated from this data. This is done by subtracting each of the post-exposure VCC values from the pooled, initial MVCCᵢ for each strain. MVCCᵢ calculations are based on plate counts for growth controls that are determined before exposure of biofilms to antimicrobials, and this calculation is carried out using equation (2). This approach is used to normalize cell death calculations to the starting number of cells as well as to average out sampling error. These calculations may be represented by the equations:

$$\begin{matrix}LK = {MVCC}_{i} - VCC & ( 4 ) \\ MLK = \sum \frac{LK}{n} = \frac{( {MVCC}_{i} - VCC )}{n} & ( 5 )\end{matrix}$$

Note that if these calculations have been performed correctly, SD will be equal in both the MVCC and MLK calculations.

### Example 15

### Calibrating a Rocking Table

If it is necessary to use a trough for biofilm cultivation on peg lids, set up a rocking table. It is crucial that the motion of this device is both (i) symmetrical and (ii) set between ˜9 and 16° of inclination. This range of motion is intended as a guideline and may vary by manufacturer. Nonequivalent biofilm formation is empirically tested as part of the protocol (Example 13), and hence, slight deviation from this recommended range might still produce acceptable results. It is possible to measure the motion of the rocker using a laser pointer, meter stick and pencil. Attach the laser pointer to the midpoint of the rocker and set the laser pointer and the rocker at a 90° angle to the wall. Use the meter stick to measure (i) the distance between the midpoint and (ii) the distance through which the laser light travels along the wall. Using trigonometry, quantify the angle through which the rocker moves. If the angle of inclination is outside the performance range or if it is asymmetrical, it will be necessary to adjust the rocker according to the manufacturer's directions. One should note that no equivalent calibration step is required for using an orbital shaker when biofilms are cultivated on peg lids that have been inserted into inoculated microtiter plates.

Asymmetrical rocking motion will cause pegs on one side of the device to be immersed to a greater depth in the inoculum than those on the other side. By contrast, a large rocking angle will cause pegs on outer rows to be submerged to a greater depth than those on the interior of the device. Either instance can lead to nonequivalent biofilm formation. Shallow rocking angles may lead to poor mixing of the growth medium in the trough and this may inhibit biofilm formation by some microorganisms. In contrast, large rocking angles can cause growth medium to slosh out of the trough. This calibration step is carried out to identify an acceptable setting for the equipment at hand.

### Example 16

### Setting Up an Incubator for Biofilm Growth

Ensure that the incubator is large enough to accommodate the orbital shaker or the rocking table used during biofilm cultivation. There should be enough space on either side of the platform for it to remain in motion unimpeded by the sides or the door of the incubator. Humidify the incubator before use by filling a tray with water and placing it on the shelf above the heating element.

### Example 17

### Biofilm Disruption Using an Ultrasonic Cleaner (“Sonicator”).

Adjust the water levels to that suggested by the manufacturer immediately before starting sonication. This ensures efficient disruption of the biofilms into the recovery medium. As the water in these devices is exposed to air, evaporation can cause significant changes in water levels in between uses. The peg lid and the microtiter plate containing recovery medium must be placed in a dry, steel tray in the ultrasonic cleaner and not directly into the water bath. Submerging the peg lid will result in contamination.

### Example 18

### Growing Microbial Cultures and Preparing Standardized Inocula for Biofilm Cultivation

FIG. 1, Step 1: grow microbial cultures. Different methods for the growth of starter cultures and preparation of the inoculum can be used. If working with cryogenic stocks from a laboratory archive, we recommend using the method of direct colony suspension from agar subcultures (option A). If working with microbial strains that have been directly isolated from a clinical or environmental specimen or if one prefers to work with liquid media, then a broth culture method could be used (option B).

### (Option A) Colony Suspension Method•TIMING ˜15 min Per Isolate on Day 3

(i) Starting from a cryogenic stock and using aseptic technique, use a sterile inoculation loop to streak out a first subculture of the desired microbial strain on the appropriate agar medium. Incubate this first subculture using the optimum growth conditions of the test organism.

Do not grow the test organisms in an antibiotic selection medium. Antibiotics can initiate adaptive stress responses in microorganisms and in some instances may lead to accumulation of mutants in the microbial population. These events can affect biofilm formation and susceptibility determinations. For many microorganisms, it is possible to grow a first subculture, to wrap it with Parafilm and to store it for up to 7 d at 4° C.

(ii) Using standard aseptic technique, use an inoculation loop to pick a single colony from the first agar subculture and then streak out a second subculture on the appropriate agar medium. Incubate this second subculture using the optimum growth conditions of the test organism. Second agar subcultures should be pure monocultures and only single colony morphology should be present.

(iii) Pipette 1.5 ml of rinse solution into a sterile glass test tube.

(iv) Use a sterile cotton swab to collect microbial colonies from the surface of a fresh second subculture. Dip the cotton swab into the rinse solution to suspend the microbes.

(v) Visually match the OD of this suspension to the appropriate McFarland standard. Comparison against a white background with contrasting black lines is helpful. If the turbidity is too high, adjust the suspension by adding sterile rinse solution; alternatively, if the turbidity is too low, add more microbial material. Use gentle vortex mixing to ensure that there are no microbial clumps in the McFarland standard suspension. Alternatively, pipette the mixture up and down using a 1,000 μl micropipette with a tip.

(vi) Pipette 29 ml of the appropriate broth growth medium into a sterile 50 ml conical tube. To this medium, add 1.0 ml of the bacterial suspension that was matched to the McFarland standard. This standardized bacterial culture serves as the inoculum for biofilm cultivation. After creating the standardized inoculum, the microbial suspension should be used within 30 min, as the cell number will begin to increase.

### (Option B) Broth Culture Method•TIMING ˜15 min Per Isolate on Day 2

(i) Starting from a agar subculture provided by a clinical or diagnostic laboratory or starting from a cryogenic stock that has been streaked out on a first agar subculture as described in Step 1(i) above, use a sterile inoculation loop or cotton swab to aseptically transfer three to five colonies from the fresh agar plate into 3-5 ml of the appropriate broth growth medium.

(ii) Incubate the broth in a shaker set at 225 rpm using the optimum growth temperature of the test organism. Grow the culture to an OD that is equal to or greater than the turbidity of the desired McFarland standard.

(iii) Transfer 1.5 ml of this culture into a sterile glass test tube.

(iv) Visually match the OD of this suspension to the appropriate McFarland standard. Comparison against a white background with contrasting black lines is helpful. If the turbidity is too high, adjust the suspension by adding sterile rinse solution; alternatively, if the turbidity is too low, add more microbial material.

(v) Pipette 29 ml of the appropriate broth growth medium into a sterile 50 ml conical tube. To this medium, add 1.0 ml of the bacterial suspension that was matched to the McFarland standard. This standardized bacterial culture serves as the inoculum for biofilm cultivation. After creating the standardized inoculum, the microbial suspension should be used within 30 min, as the cell number will begin to increase.

### Example 19

### Biofilm Cultivation•TIMING ˜40 min Per Isolate on Day 1

FIG. 1, Step 2| Next cultivate biofilms. There are at least two approaches that may be used to grow biofilms on peg lids: biofilms may be grown on peg lids inserted into inoculated microtiter plates (option A) or on peg lids inserted into inoculated troughs (option B). If pegs lids need to be surface modified, then this should be carried out ahead of time (see Example 11).

### (A) Growing Biofilms on Peg Lids in Microtiter Plates

(i) In a biological safety cabinet, open the sterile packages containing the peg lid reactor(s) and a reagent reservoir.

(ii) Pour the standardized inoculum prepared in Step 1 into a reagent reservoir.

(iii) Using the multichannel pipette, add 150 μl of the standardized inoculum to each well of the microtiter plate. Do not use more than 150 μl of inoculum in each well. This volume ensures that the biofilms grown on the peg surfaces will be completely submersed in the rinse and antimicrobial challenge plates prepared in subsequent steps of the protocol.

(iv) Insert the peg lid into the wells of the microtiter plate. To ensure the correct fit, peg A1 of the device should be inserted into well A1 of the microtiter plate.

(v) Seal the inoculated device with Parafilm.

(vi) Place the inoculated device on an orbital shaker in a humidified incubator. Select an appropriate speed of rotation and temperature that correspond to the optimum for the test organism and incubate the inoculated device for an appropriate period of time (e.g., see Example 28).

### (B) Growing Biofilms on Peg Lids in Troughs

(i) In a biological safety cabinet, open the sterile packages containing the peg lid reactor(s).

(ii) Pipette 22 ml of the standardized inoculum prepared in Step 1 into the corrugated trough.

(iii) Insert the peg lid into the trough.

(iv) Seal the inoculated device with Parafilm.

(v) Place the inoculated device on a rocking table in a humidified incubator. Select an appropriate rocking speed and temperature that correspond to an optimum for the test organism. Incubate the inoculated device for an appropriate period of time (e.g., see Example 28).

### Example 20

### Verify the Starting Cell Number in the Standardized Inoculum•TIMING ˜20 min Per Isolate on Day 2

(FIG. 1, Steps 3-8) 3| In a biological safety cabinet, open a sterile package containing a reagent reservoir. Pipette 10 ml of rinse solution into the reservoir.

4| For each standardized inoculum prepared, pipette 180 μl of sterile rinse solution into four columns of wells, from rows A to F, of a sterile 96-well microtiter plate.

5| (Transfer a 20 μl aliquot of the standardized inoculum into row A of each column.

6| Use a multichannel micropipette to serially dilute these 20 μl aliquots in the sterile rinse solution. To do this, mix the contents of row A by pipetting up and down, then transfer 20 μl from the wells of row A into the corresponding wells of row B. Repeat this serial mix and transfer process for each of the remaining rows B to F, each time taking a 20 μl aliquot from the most recently prepared dilution and transferring it to the next row in the series.

Change the tips of the multichannel pipette between each mix and transfer step. This prevents incidental carry-over of bacteria from the lowest to highest dilutions.

7| Using a multichannel pipette, transfer 10 μl aliquots from every well of rows F to A of the microtiter plate onto the surface of the appropriate agar growth medium. Incubate this ‘spot’ plate using the optimum growth conditions of the test organism.

It is possible to carry out this step without changing tips on the micropipette. To do this, the 10 μl spots are applied in order from the highest (10⁻⁸, row F) to lowest (10⁻¹, row A) dilutions.

8| Score the spot plates for growth by colony counting. To ensure accurate colony counts, score the colonies at the lowest test dilution at which it is still possible to count them. Determine the mean VCC and SD for the inoculum using equations (2) and (3).

The mean VCC determined from these controls should be within 1.0 log₁₀CFU ml⁻¹ of the desired starting cell number. This ensures reproducible biofilm growth from one experiment to the next. Devices inoculated with cell numbers outside the target range should be discarded along with any data generated from these assays. The rationale is to eliminate the potential contribution of an inoculum effect to biofilm growth and to the subsequent antimicrobial susceptibility determinations.

### Example 21

### Set Up an Antimicrobial Challenge Plate•TIMING ˜60 min Per Isolate on Day 1

(FIG. 1, Steps 9-15) 9| If this is the first time that biofilms have been cultivated under these test conditions, then check for asymmetry in biofilm formation on the peg lid before undertaking any subsequent step experiments for susceptibility testing (Example 13). If biofilm cultivation conditions are sufficient, then proceed to make up a 2 ml working solution for each of the desired antimicrobial agents in the appropriate growth media. Do not dilute the growth medium by using more than one part stock antimicrobial solution per four parts of growth medium. The working solution of the antimicrobial should be made at a concentration equal to the highest concentration to be tested in the challenge plate.

10| In a biological safety cabinet, open sterile packages containing one 96-well microtiter plate and three reagent reservoirs. Pipette 25 ml of growth medium into one reservoir and add the working solutions of the antimicrobial agents into the others.

11| Using a multichannel pipette, transfer 200 μl of growth medium into the wells of columns 1 and 12 of the microtiter plate. Leaving the wells in column 2 empty for the time being, transfer 100 μ_{l of growth medium to all the wells of columns} 3-11 in the microtiter plate.

As previously noted, one can set up challenge plates in any desired configuration and this is not limited to the alternating, log₂ dilution gradient of two antimicrobials presented here. However, it is crucial to include growth and sterility controls in every challenge plate. In the present step, the wells of columns 1 and 12 serve as the sterility and growth controls, respectively.

12| Using a multichannel pipette, transfer 200 μl of the first antimicrobial working solution into the wells A2, C2, E2, G2 and 100 μl into the wells A3, C3, E3, G3 and A4, C4, E4 and G4. Next, transfer 200 μl of the second antimicrobial working solution into the wells B2, D2, F2, H2 and 100 μl into the wells B3, D3, F3, H3 and B4, D4, F4 and H4. This sets up the challenge plate so that dilutions of each antimicrobial are made in alternating rows of the microtiter plate.

Change pipette tips between handling the different antimicrobials. This ensures that there is no cross-contamination of the two agents.

If the quantity of antimicrobial is limited, it is possible to use a single channel 200 μl micropipette to arrange the working solution in the antimicrobial challenge plate.

13| Serially dilute 100 μl aliquots of the antimicrobial working solution in the growth medium. To do this, transfer 100 μl of growth medium to column 4 and then mix the contents by pipetting up and down. Transfer 100 μl from the wells of column 4 into the corresponding wells of column 5. Repeat this serial mix and transfer process for each of the remaining columns 5-11, each time taking a 100 μl aliquot from the most recently prepared dilution and transferring it to the next column of wells in the series. After the contents of column 11 have been mixed thoroughly, extract 100 μl from each well of column 11 and discard.

14| Using a multichannel pipette, add 100 μl of growth medium to every well in columns 4-11. The final volume of every well in the challenge plate should now be 200 μl.

To prevent backward contamination of the growth medium, do not insert the pipette tips into the challenge media. Instead, lean the sides of the pipette tips against the edges of the microtiter plate wells. This allows the lumen of the tip to be suspended in the air above the center of each well and prevents direct contact of the tips with the antimicrobial agents below.

The final volume in each well of the antimicrobial challenge plate should be 200 μl. Volumes less than this may be insufficient to completely immerse the biofilms; in contrast, volumes greater than this might overflow when the peg lid is inserted into the wells.

15| Replace the sterile lid on the microtiter plate and place this challenge plate aside for use in Step 24. Antimicrobial challenge plates should be used within 1 h of preparation.

### Example 22

### Controls for Batch Culture Growth of Microbes in the Peg Lid Reactor and Exposure of Biofilms to Antimicrobials•TIMING ˜30 min per Isolate on Day 2

(FIG. 1, Steps 16-31) 16| In the biological safety cabinet, open sterile packages containing three 96-well microtiter plates and two reagent reservoirs. Pipette 25 ml of rinse solution into one reservoir and 5 ml of recovery medium into the other. Refill the reagent reservoirs as required during Step 17.

17| Using a multichannel pipette, add 200 μl of rinse solution to each well of the first microtiter plate. This first plate will be used in Step 19 to rinse the biofilms. To the second plate, add 200 μl of recovery medium to wells A1, B1, C1 and D1, and then add 180 μl of rinse solution to wells in columns 1, 2, 3 and 4. This second plate will be used in Step 21 to verify batch culture biofilm cell counts. To the third plate, transfer 180 μl of rinse solution into each well of columns 1-4. This last plate will be used in Step 26 to determine batch culture planktonic cell counts.

18| Ignite the ethanol lamp. As a general rule, it is recommended that open flames should not be used in biological safety cabinets. Extra care must be taken as flames in laminar airflow can move rapidly. In addition, take extra care to not to ignite clothing or latex gloves that may have come into contact with ethanol while working in the biological safety cabinet.

19| Rinse the biofilms formed in Step 2 by submersing the peg lid into the wells of the microtiter plate containing the rinse solution. Let them stand for 1 min.

20| Dip the tips of the needle nose pliers into the jar filled with 95% ethanol and then flame them using ethanol lamp.

21| Remove the peg lid from the rinse solution. Using needle nose pliers, break off pegs A1, B1, C1 and D1 and place them into wells A1, A2, A3 and A4 of the second microtiter plate prepared in Step 17. When removing a peg from the lid, grab the peg by positioning the pliers as close to the lid as possible. Ensure that the pliers do not touch an area on the peg in which the biofilm is growing. Pull the peg off the lid in a direction away from any other pegs in proximity, ensuring that no other peg is touched in the process. Pegs should always be removed from outer rows toward the inside rows to prevent scraping of adjacent peg surfaces. The needle nose pliers should be flamed each time a new peg is removed from the device.

22| Break off the remaining pegs in column 1 of the device and discard them in appropriate biohazardous waste container.

23| Extinguish the ethanol lamp.

24| Place the peg lid into the challenge medium prepared in Step 15. Seal the plate with Parafilm and incubate at the required temperature for the desired exposure time.

25| Place the microtiter plate containing the broken pegs onto the metal insert tray of the ultrasonic cleaner. Disrupt the biofilms into the recovery medium by sonicating for 10 min.

26| While the biofilms are being disrupted, return to the biological safety cabinet. Using a multichannel pipette, transfer a 20 μl aliquot of the now turbid inoculum into each of the wells A1, B1, C1 and D1 of the third microtiter plate prepared in Step 17. If biofilms were grown in a trough format device, these four aliquots are taken from the fluid sitting in the bottom of the trough; if biofilms were grown in microtiter plates, these four aliquots can be taken from any four different wells.

27| Discard the planktonic inoculum into an appropriate biohazardous waste receptacle.

28| Use a multichannel micropipette to serially dilute 20 μl aliquots of the planktonic cells suspended in row A of the microtiter plate prepared in Step 26. Use the same technique described in Step 6 above.

29| Retrieve the microtiter plate containing the broken pegs from the sonicator and return to the biological safety cabinet. Use a multichannel micropipette to serially dilute 20 μl aliquots from the recovery medium, which contains the disrupted biofilm cells, in the sterile rinse solution. Use the same technique described in Step 6 above.

30| Using a multichannel pipette, transfer 10 μl aliquots from every well of rows F to A of the microtiter plates from Steps 28 (planktonic cells) and 29 (biofilm cells) onto the appropriate agar growth medium. Use the same technique described in Step 7 above. Incubate these ‘spot’ plates using the optimum growth conditions of the test organism.

31| Score the spot plates for growth by colony counting using the approach described in Step 8. Calculate the MVCC, and SD using equations (2) and (3).

### Example 23

### Rinse and Recover Biofilms•TIMING ˜15 min per Isolate on Day 1

(FIG. 1, Steps 32-38) 32| In a biological safety cabinet, open sterile packages containing two reagent reservoirs and three 96-well microtiter plates. Pipette 25 ml of rinse solution into one reagent reservoir and 25 ml of recovery medium into the other. Refill the reagent reservoirs as a required.

33| Using a multichannel pipette, transfer 200 μl of rinse solution into every well of the first two microtiter plates. Next, transfer 200 μl of recovery medium into every well of the third microtiter plate.

34| Retrieve the challenge plate, which contains the exposed biofilms, from the incubator and place it in the biological safety cabinet.

35| Remove the peg lid from the challenge medium and submerse the pegs into rinse solution in the first rinse plate prepared in Step 33. Let them stand for 1 min.

36| Remove the peg lid from the first rinse plate and submerse the pegs into rinse solution in the second rinse plate prepared in Step 33. Let them stand for an additional 1 min.

37| Remove the peg lid from the second rinse plate and submerse the pegs into the recovery medium in the third microtiter plate prepared in Step 33. Place the microtiter plate containing the peg lid onto the metal insert tray of the ultrasonic cleaner. Disrupt the biofilms into the recovery medium by sonicating for 10 min.

38| Discard the microtiter plates containing the challenge medium and spent rinse solution in appropriate biohazardous waste container. Retain the sterile lid from the microtiter plate containing the recovery medium.

### Example 24

Determine End Points from Antimicrobial Susceptibility Testing

(FIG. 1, Step 39) 39| Determine biofilm susceptibility to antimicrobials qualitatively (option A) or qualitatively and quantitatively (option B).

### (Option A) Qualitative End Point Determinations•TIMING ˜5 min per Test Isolate on Day 2

(i) When sonication is completed, retrieve the peg lid and the recovery plate and return to the biological safety cabinet. Remove the peg lid from microtiter plate and cover the recovery medium with the lid retained in Step 38. Discard the peg lid in appropriate biohazardous waste container.

(ii) Seal this microtiter plate with Parafilm. Incubate this recovery plate for a suitable period of time using the optimum growth conditions of the test organism.

(iii) Read the OD of the microtiter plate wells at 650 nm using the microtiter plate reader and visually inspect the wells for growth. The MBEC corresponds to the lowest of an antimicrobial agent that results in no visual growth of microbes in the well of the microtiter plate (see Example 25).

### (Option B) Qualitative and Quantitative Determinations of Survival•TIMING ˜90 min Per Isolate on Day 2

(i) While the biofilms are being disrupted by sonication, return to the biological safety cabinet and open sterile packages containing eight microtiter plates. Using a multichannel pipette, transfer 180 μl of rinse solution into rows B to F of each of these microtiter plates.

(ii) When sonication is completed, retrieve the peg lid and the recovery plate and return to the biological safety cabinet. Remove the peg lid from microtiter plate and cover the recovery medium with the lid retained in Step 38. Discard the peg lid in appropriate biohazardous waste container.

(iii) Using a multichannel pipette, transfer 50 μl of the recovery medium from row A of the recovery plate to row A of one of the microtiter plates. Repeat this transfer process for each row of the recovery plate, pipetting 50 μl of the recovery medium into row A of a separate microtiter plate prepared in Step 39B(i).

(iv) Using a multichannel pipette, serially dilute 20 μl aliquots from the recovery medium, taken from row A of these microtiter plates, into the sterile rinse solution. Use the same technique described in Step 6.

(v) Using a multichannel pipette, transfer 10 μl aliquots from every well of rows F to A of each of these microtiter plates onto the appropriate agar growth medium. Use the same technique described in Step 7. Incubate this spot plate using the optimum growth conditions of the test organism.

(vi) Seal the microtiter plate containing the recovery medium with Parafilm and incubate as described in Step 39A(iii).

(vii) Read the OD of the microtiter plate wells as described in Step 39A(iii) and determine the MBEC values.

(viii) Score the spot plates for growth by colony counting using the same approach described in Step 8. Determine the mean VCCs and SD for the broth culture using equations (2) and (3). Calculate biofilm mean log-killing and SD using equation (5).

### Example 25

To illustrate the results obtained through this protocol, Biofilms of P. aeruginosa ATCC 27853, a control strain used routinely in standardized CLSI testing, were grown in LB broth using the parameters outlined in Example 28. The biofilms were then exposed to gentamicin for either 2 or 20 hours; qualitative and quantitative determinations of biofilm cell survival were then performed. For ease of understanding, we focus on a single antibiotic.

Mean VCC for the Standardized Inoculum (Steps 3-8)

In most cases, reproducible biofilm growth occurs when the inoculum is prepared to within 1.0 log₁₀ of the target cell number, which in this case was 7.0 log₁₀ CFU ml⁻¹ (Example 28), and this corresponded to a 30-fold dilution of a McFarland standard 1.0. Here, we inoculated three devices; one was used to perform a test for equivalent growth, and two were used on a subsequent day to carry out susceptibility determinations. We took three or four measurements to verify the size of these inocula, and on average, the starting cell number was 7.4±0.2, 7.1±0.3 and 7.0±0.2 log₁₀ CFU ml⁻¹, respectively.

### Test for Nonequivalent Biofilm Formation (Example 13)

We enumerated bacteria on 48 pegs of one device and grouped these data by row. Note that these counts were ascertained from pegs still attached to the peg lid when they were sonicated into recovery medium in a microtiter plate. VCCs were 6.5±0.2, 6.5±0.2, 6.5±0.1, 6.4±0.4, 6.2±0.6, 6.1±0.6, 6.2±0.5 and 6.3±0.5 log₁₀ CFU per peg for rows A to H, respectively. When tested by one-way ANOVA, there was no significant difference in population means in different rows of the device (P=0.842)⁴⁹.

### Controls for Batch Culture Growth of Microbes (Steps 16-31)

Viable cell counting indicated a mean population density of 9.1±0.3, 9.0±0.2 and 9.2±0.2 log₁₀ CFU ml⁻¹ for planktonic microbes growing in the troughs of each of these devices. In the case of the devices used for susceptibility testing, the biofilm MVCCᵢ was 6.5±0.2 and 6.6±0.2 log₁₀ CFU per peg for the devices used for 2 and 20 h exposure measurements, respectively. Note that these biofilm counts were determined from pegs broken from the lid before they were sonicated into the recovery medium. Thus, biofilm cell numbers were similar regardless of whether pegs remained attached or were broken from the lid before sonication (i.e., these counts were directly comparable with the results obtained from the test for equivalent biofilm formation above).

### Determinations of Biofilm Susceptibility to Antimicrobials (Step 39)

Whether exposed for 2 or 20 hours to gentamicin, we determined that the MBEC was >512 μg ml⁻¹ for P. aeruginosa ATCC 27853 biofilms. One should note that it is normal for cells to be lost from biofilms during the process of antimicrobial exposure; however, these cells will recommence growth during an appropriate incubation time, and this feature of the peg lid system allows the MBIC to be determined (FIG. 8). Here, VCC indicated an MBIC=128 μg ml⁻¹ gentamicin, which corresponded to the point wherein biofilm cell numbers no longer increased during exposure. The MBCB was >512 μg ml⁻¹ gentamicin when biofilms were only exposed for 2 h; however, the MBCB was 512 μg ml⁻¹ gentamicin after 20 h exposure.

### Example 26

(a) A batch culture biofilm reactor of the present invention may comprise a plastic lid with 96 pegs that is covered with an adhesive backing. The backing allows pegs to be detached from the lid without compromising the integrity of the device. The peg lid can be fit into a standard 96-well microtiter plate or into a grooved trough, either of which can serve as the inoculum reservoir for biofilm cultivation. The lid of the reactor has a lip that fits snugly against the microtiter plate or trough, and a plastic stop prevents insertion of the pegs in the reverse direction. (b) Each peg has a total surface area of ˜109 mm². Biofilms grown using the inoculum volumes suggested in this protocol cover an area of ˜44 mm². Each peg is engineered with a break point that allows it to be removed from the lid with needle nose pliers. This break point is positioned above the anticipated air-liquid-surface interface wherein biofilm growth is at a maximum. (c) Intralaboratory reproducibility of P. aeruginosa 15442 biofilms cultivated in the CBD. This organism forms biofilms with an overall mean cell density of 5.0±0.6 log₁₀ CFU mm⁻² (or ˜6.6±0.6 log₁₀ CFU per peg) in the CBD. The repeatability standard deviation of the log density based on eight pegs per experiment was 0.74, variation of which is comparable to biofilms grown on coupons in the Center for Disease Control (CDC) biofilm reactor¹⁸. Moreover, the mean cell density from this evaluation is nearly identical to the 6.8±0.6 log₁₀ CFU per peg (n=297), previously reported in the literature for peg biofilms of P. aeruginosa ATCC 15442 grown under similar nutritional conditions¹⁹.

### Example 27

Examples of organisms previously grown in multispecies biofilms on peg lids.

| 1^{st} microorganism | 2^{nd} microorganism | 3^{rd} microorganism | Ref. |
|---|---|---|---|
| Aggregatibacter | Viellonella sp. | | 23 |
| actinomycetemcomitans | | | |
| | Viellonella sp. | Fusobacterium nucleatum | 23 |
| Fusobacterium nucleatum | Aggregatibacter | | 23 |
| | actinomycetemcomitans | | |
| | Actinomyces naeslundii | Streptococcus oralis | 22 |
| Porphyromonas gingivalis | Aggregatibacter | | 54 |
| | actinomycetemcomitans | | |
| | Fusobacterium nucleatum | | 54 |
| | Viellonella sp. | | 54 |
| Pseudomonas aeruginosa | Burkholderia cenocepacia | | 55 |

### Example 28

As noted in the specific description and in the examples above, the protocol shown in

FIG. 1 and described in Example 25 (among others) may be changed or tailored for a specific microorganism. Typically the changes are intended to improve biofilm growth, improve biofilm/bacteria adherence to the substrate or pegs, improve the quality of the results in the susceptibility testing, and/or to address nutritional or environmental cues for a specific species.

The inventors have established amended protocol parameters and apparatus configurations for more than 65 different microbial species. These are listed below in table 4.

TABLE
Batch culture parameters and apparatus configurations for microbial attachment and/or biofilm growth on peg lids.

| | | Lid | Motion′ | T | Time | Inoculum³ | Growth | Atmospheric | Mean cell count | | |
|---|---|---|---|---|---|---|---|---|---|---|---|
| Genus and species | Strain | surface′ | (rpm) | (° C.) | (h) | (CFU/ml) | Medium⁴ | gases | (log₁₀CFU/peg) | n⁶ | Ref. |
| Actinomyces | ATCC | N, human | static | 37 | 24 | 1.0 × 10⁷ | 25% | N₂/CO₂/H_{2,} | −6.5 ± 0.2 | 3 | 1 |
| naeslundii | 43146 | saliva | | | | | human | 90:5:5 | | (qPCR) | |
| | | coated | | | | saliva | | | | | |
| Acinetobacter | undesignated | M | O (110) | 37 | 20 | 1.0 × 10⁷ | TSB | ambient | 6.8 ± 0.1 | 5 | [symbol] |
| baumannii | clinical | | | | | | | | | | |
| | isolate | | | | | | | | | | |
| Aggregatibacter | JP2 | N, human | static | 37 | 36 | 1.0 × 10⁷ | 25% | N₂/CO₂/H₂, | −7.0 ± 0.1 | 3 | 2 |
| actinomycetemcomita | | saliva | | | | | human | 90:5:5 | | (qPCR) | |
| | | coated | | | | | saliva | | | | |
| Alcaligenes | ATCC | M | O (110) | 37 | 20 | 1.0 × 10⁷ | TSB | ambient | 6.9 ± 0.4 | 6 | [symbol] |
| xylosoxidans | 55564 | | | | | | | | | | |
| Arcanobacterium | veterinary | M | R (10) | 37 | 24 | 1.0 × 10⁷ | TSB + | air/C0₂, | 5.0 | NR | 3 |
| pyogenes | isolate | | | | | | 2% FCS | 90:10 | | | |
| Burkholderia | J2315 | M | O (125) | 37 | 24 | 1.0 × 10⁷ | CA-MHB | ambient | −4.3 ± 0.4 | 6 | 4 |
| cenocepacia | | | | | | | | | | | |
| | K56-2 | M | O (125) | 35 | 72* | 1.0 × 10⁷ | MSD-YC | ambient | 6.6 ± 0.4 | 4 | 5 |
| Campylobacter jejuni | veterinary | M | R (3.5) | 37 | 24 | 1.0 × 10⁷ | CA-NM | air/C0₂, | 4.8 ± 0.1 | 10 | [symbol] |
| | isolate | | | | | | | 90:10 | | | |
| Candida albicans | 3153A | M, TCA + | O (75) | 35 | 48 | 1.0 × 10⁵ | RQMB | ambient | 2.9 ± 0.5 | 4 | 6 |
| | | EtO | | | | | | | | | |
| | ATCC | M | R (NR) | 35 | 72 | 1.0 × 10⁷ | artificial | ambient | −7.0 ± 0.4 | 9 | 7 |
| | 14053 | | | | | | test soil | | | | |
| | ATCC | N | O (75) | 37 | 51** | 1.0 × 10⁷ | YNB | ambient | NR (XTT) | NA | 8 |
| | 90028 | | | | | | | | | | |
| Candida dubliniensis | ATCC | N | O (75) | 37 | 51** | 1.0 × 10⁷ | YNB | ambient | NR (XTT) | NA | 8 |
| | MYA646 | | | | | | | | | | |
| Candida glabrata | ATCC 2001 | N | O (75) | 37 | 51** | 1.0 × 10⁷ | YNB | ambient | NR (XTT) | NA | 8 |
| Candida krusei | ATCC 6258 | N | O (75) | 37 | 51** | 1.0 × 10⁷ | YNB | ambient | NR (XTT) | NA | 8 |
| Candida tropicalis | 99916 | M, | O (125) | 35 | 48 | 1.0 × 10⁵ | TSB, | ambient | 4.3 ± 0.4, 4.4 ± 0.3 | 168.4 | 6, 9 |
| | | L-lysine | | | | | RQMB | | | | |
| | | coated | | | | | | | | | |
| | ATCC | N | O (75) | 37 | 51** | 1.0 × 10⁷ | YNB | ambient | NR (XTT) | NA | 8 |
| | 13803 | | | | | | | | | | |
| | NCTC 7393 | M | O(125) | 37 | 24 | 1.0 × 10⁷ | CA-MHB | ambient | 5.0 ± 0.1 | 6 | 4 |
| Clostridium difficile | ATCC 9689 | M | O(110) | 37 | 48 | 1.0 × 10⁶ | BH1 | anaerobic | 3.7 ± 0.3 | 10 | [symbol] |
| Pseudomonas | ATCC | M | O(100) | 27 | 48 | 1.0 × 10⁷ | LB | ambient | 6.0 ± 0.8 | 99 | 30 |
| fluorescens | 13525 | | | | | | | | | | |
| Pseudomonas | PTB2093 | M | O (150) | 15 | 48 | 1.0 × 10⁷ | maple sap | ambient | −7.4 ± 0.1 | 5 | 31 |
| marginalis | | | | | | | | | | | |
| Pseudomonas | KF707 | M | O (100) | 30 | 24 | 1.0 × 10⁷ | LB, SA | ambient | 5.4 ± 0.2-6.2 ± 0.5 | 16 | 32 |
| pseudoalcaligenes | | | | | | | | | | | |
| Rhizobium | 3841 | M | R (3.5) | 30 | 72 | 1.0 × 10⁷ | VMM + | ambient | 5.3 ± 0.5 | 104 | 33, |
| leguminosarum | | | | | | | 1% | | | | 34 |
| | | | | | | | mannitol | | | | |
| Salmonella enteric | veterinary | M | R (10) | 37 | 6 | 1.0 × 10⁷ | TSB | ambient | 6.0 | NR | 3 |
| serovar Bredenay | isolate | | | | | | | | | | |
| Salmonella enteric | ATCC | M | O (125) | 37 | 24 | 1.0 × 10⁷ | CA-MHB | ambient | 4.7 ± 0.4 | 64 | 24 |
| serovar Cholerasuis | 10708 | | | | | | | | | | |
| | | M | R (3.5) | 37 | 24 | 1.0 × 10⁷ | TSB | ambient | 5.8 ± 0.2 | 10 | [symbol] |
| Salmonella enteric | SL1344 | N | static | 16 | 48 | 1.0 × 10⁷ | 5% TSB | ambient | NR (CV) | NA | 35 |
| serovar | | | | | | | | | | | |
| Sphingomonas | EPA505 | N, PA | O (50) | 25 | 72 | 1.0 × 10⁷ | BMM | ambient | NR (CV) | NA | 36 |
| paucimobilus | | coated | | | | | | | | | |
| Sphingomonas | B1 | N | O (50) | 25 | 72 | 1.0 × 10⁷ | R2A | ambient | NR (CV) | NA | 36 |
| yanoikuyae | | | | | | | | | | | |
| Sphingopyxis sp. | TP340-8 | N | O (50) | 25 | 72 | 1.0 × 10⁷ | R2A | ambient | NR (CV) | NA | 36 |
| Staphylococcus | ATCC | N | R (3.5) | 35 | 24 | 1.0 × 10⁷ | LB | ambient | 6.2 ± 0.9 | NR | 11 |
| MSSA | 29213 | | | | | | | | | | |
| | | M | O (150) | 37 | 72* | 1.0 × 10⁷ | TSB | ambient | 5.0 ± 0.1 | 3 | 37 |
| | ATCC 6538 | M | O (125) | 37 | 24 | 1.0 × 10⁷ | TSB | ambient | 5.6 ± 0.4 | 60 | 24 |
| MRSA | E-MRSA 15 | M | O (125) | 37 | 24 | 1.0 × 10⁷ | CA-MHB | ambient | −5.2 ± 0.4 | 6 | 4 |
| | MRSA 201 | M | O (125) | 37 | 24 | 1.0 × 10⁷ | CA-MHB | ambient | −5.2 ± 0.3 | 6 | 4 |
| | ATCC | M | O (150) | 37 | 24 | 1.0 × 10⁷ | TSB | ambient | 5.0 ± 0.1 | 3 | 37 |
| | 33591 | | | | | | | | | | |
| VRSA | YRS5 | M | O (150) | 37 | 24 | 1.0 × 10⁷ | TSB | ambient | 5.0 ± 0.1 | 3 | 37 |
| Staphylococcus | ATCC | M | O (125) | 37 | 24 | 1.0 × 10⁷ | CA-MHB | ambient | −5.6 ± 0.1 | 6 | 4 |
| epidermidis | 35984 | | | | | | | | | | |
| Staphylococcus | veterinary | M | R (10) | 37 | 7.5 | 1.0 × 10⁷ | TSB | air/C0₂, | 5.8 | NR | 3 |
| hyicus | isolate | | | | | | | 90:10 | | | |
| Staphylococcus | undesignated | N | static | 37 | 24 | 4.0 × 10⁶ | TSB + 1% | ambient | NR (OD) | NA | 38 |
| lugdunensis | clinical | | | | | | glucose | | | | |
| | isolates | | | | | | | | | | |
| Stenotrophomonas | ATCC | M | O(110) | 37 | 20 | 1.0 × 10⁷ | NB | ambient | 6.4 ± 0.2 | 6 | [symbol] |
| maltophilia | 12714 | | | | | | | | | | |
| Streptococcus | veterinary | M | R (10) | 37 | 7 | 1.0 × 10⁷ | TSB | air/C0₂, | 5.2 | NR | 3 |
| agalactiae | isolate | | | | | | | 90:10 | | | |
| Streptococcus bovis | veterinary | M | R (10) | 37 | 7 | 1.0 × 10⁷ | TSB | air/C0₂, | 5.2 | NR | 3 |
| | isolate | | | | | | | 90:10 | | | |
| Streptococcus | ATCC | M, HA | O (110) | 37 | 48 | 1.0 × 10⁶ | THB | air/C0₂, | 6.6 ± 0.1 | 6 | [symbol] |
| cristatus | 49999 | coated | | | | | | 90:10 | | | |
| Streptococcus | veterinary | M | R (10) | 37 | 7 | 1.0 × 10⁷ | TSB | air/C0₂, | 5.0 | NR | 3 |
| dysgalactiae | isolate | | | | | | | 90:10 | | | |
| Streptococcus | undesignated | N, human | static | 37 | 24 | 1.0 × 10⁷ | 25% | N₂/C0₂/H₂, | −5.8 ± 0.1 | 3 | 39 |
| gordonii | clinical | saliva | | | | | human | 90:5:5 | | (qPCR) | |
| | isolate | coated | | | | | saliva | | | | |
| | DLI | M, horse | O (100) | 37 | 20 | O/N | BHI + 5% | ambient | NR (CV) | NA | 40 |
| | | serum | | | | | sucrose | | | | |
| | | coated | | | | | | | | | |
| Streptococcus | ATCC | M | static | 37 | 24 | 1.0 × 10⁶ | BHIS | anaerobic | NR (CV) | NA | 4′ |
| mutans | 10449 | | | | | | | | | | |
| | ATCC | M, HA | O (110) | 37 | 48 | 1.0 × 10⁶ | M260 | air/C0₂, | 6.7 ± 0.1 | 6 | [symbol] |
| | 700610 | coated | | | | | | 90:10, | | | |
| Streptococcus oralis | 34 | N, human | static | 37 | 24 | 1.0 × 10⁷ | 25% | N₂/C0₂/H₂, | −6.0 ± 0.1 | 3 | 1 |
| | | saliva | | | | | human | 90:5:5 | | (qPCR) | |
| | | coated | | | | | saliva | | | | |
| Streptococcus | ATCC | M | O (110) | 37 | 20 | 1.0 × 10⁷ | M260 | ambient | 4.6 ± 0.2 | 6 | [symbol] |
| pneumoniae | 10015 | | | | | | | | | | |
| Streptococcus suis | veterinary | M | R (10) | 37 | 7 | 1.0 × 10⁷ | TSB | air/C0₂, | 4.8 | NR | 3 |
| | isolate | | | | | | | 90:10 | | | |
| Veillonella sp.≠ | PK1910 | N, human | static | 37 | 36 | 1.0 × 10⁷ | 25% | N₂/C0₂/H₂, | −5.5 ± 0.3 | 3 | 2 |
| | | saliva | | | | | human | 90:5:5 | | (qPCR) | |
| | | coated | | | | | saliva | | | | |

′Denotes the supplier of the peg lid and any surface modification used to grow the test organism on the peg surfaces: HA = hydroxyapetite; M = MBEC assay lid; N = Nunc Immuno-TSP solid surface ELISA lid; PA = phenanthrene.
²Denotes the assay format and the rate of rocking or orbital motion: R = rocking table for trough format (rocks per minute); O = orbital shaker for microtiter plate format (revolutions per minute); NR = not reported.
³Denotes the cell density of the standardized inoculum for biofilm cultivation. O/N = denotes that an overnight culture was used as the inoculum.
⁴Abbreviations for growth media: ADC = albumin, dextrose and catalase enrichment; AYE = N-(2-acetamido)-2-aminoethanesulfonic acid (ACES)-buffered yeast extract medium; BHI = brain heart infusion broth; BHIC = BHI supplemented with yeast extract and L-cystine; BHIS = 25% brain-heart infusion broth supplemented with 2% sucrose; BMM = Brunner's minimal medium; CA-NM = cation adjusted Mueller Hinton broth; FCS = fetal calf serum; HTM = Haemophilus test medium; KB = King's broth; LB = Luria-Bertani broth; M260 = American Tissue Culture Collection medium 260; M1490 = American Tissue Culture Collection medium 1490; MSD = minimal salts dextrose; MSD-YC = minimal salts dextrose enriched with yeast extract and casamino acids; MSVP = minimal salts vitamins pyruvate; NB = nutrient broth; R2A = Reasoners 2A medium; RQMB = Roswell Park Memorial Institute (RPMI) 1640 supplemented with glutamine and buffered with MOPS and sodium bicarbonate; SA = sucroseasparagine medium; THB = Todd Hewitt broth; TSB = tryptic soy broth; TSB-YE, tryptic soy broth with yeast extract; VMM = Vincent's minimal medium; YNB = yeast nitrogen base medium supplemented with 100 mM glucose
⁵A ‘-’ denotes that mean cell counts were calculated from graphical data. NR = not reported, CV = denotes microbial biomass was evaluated using crystal violet staining; OD = denotes biofilm growth was evaluated qualitatively using optical density endpoints as described in this protocol; XTT = biofilm growth was evaluated using the tetrazolium salt 2,3-bis(2-methoxy-4-nitro-5-sulfo-phenyl)-2H-tetrazolium-5-carboxanilide as a metabolic indicator.
⁶n denotes the number of replicate measurements used to calculate the mean biofilm cell count and standard devistion. NA = not applicable; NR = not reported; gPCR = cell number was determined by quantitative polymerase chain reaction.
*Growth medium was changed every 24 h by transferring the peg lid to a new microtiter plate containing 150 R1 of fresh medium in each well.
**Growth medium wash change after the first 3 h of incubation by transferring the peg lid to a new microtiter plate containing 150 gl of fresh medium in each well. [symbol]
(Unpublished results, N. D. Allan and M. E. Olson.
≠These microorganisms will grow optimally in static culture as part of a multispecies biofilm

### Example 29

FIGS. 8(a, b, and c) illustrate an exemplary method of reading qualitative end points from patterns in recovery plates and interpreting biofilm survival data from kill curves.

In FIG. 8a, column 1 includes a sterility control and column 12 includes a growth control. Columns 2-11 may contain different dilutions of antimicrobial agent, e.g., as shown, x, x/2, x/4, x/8, x/16, x/64, x/128, x/256, and x/512.

FIG. 8a shows interpretations of growth patterns in recovery plates for determining MBEC endpoints, and that it is similar in many ways to MIC testing, with three exceptions. First, a skipped well, e.g., a clear well in a series of wells with visible growth, is usually ignored; however, it might indicate uneven biofilm growth in the device or that biofilms of a specific species might need to be grown longer before antimicrobial exposure.

Second, scant growth in the recovery medium might indicate low numbers of survivors in biofilms, which may be seen for some variant cell populations that characteristically arise during biofilm cultivation.

Finally, paradoxical growth, e.g., wherein several wells in the middle of a dilution series are clear, but visible growth occurs at low and high concentrations, can occur for biofilms of some microbial species, especially Candida.

The rows as illustrated are shown as examples of how growth patterns may be interpreted. Row A shows that the MBEC is greater than x, indicating , if it is relevant, that the concentration range needs to be increased and the test repeated. Row B shows that the MBEC is equal to x/32. Row C shows that the MBEC is less than or equal to x/512, and if relevant, that the concentration range needs to be decreased and the test repeated. Row D shows the MBEC equal to x, and that low numbers of biofilm survivors when the concentration is greater than x/128. Row E shows that the MBEC is greater than x or insufficient biofilm cultivation time, and that the test should be repeated. Row F shows asymmetrical biofilm formation, and that growth conditions should be optimized before the test is repeated, or that there is paradoxical killing of the microorganism by the antimicrobial agent. Row G shows that the recovery medium is likely contaminated and that the test should be repeated. Row H shows that the organism did not grow in the recovery medium and that the test should be repeated.

FIG. 8b shows the theoretical quantitative time- and concentration-dependent mean viable cell counts (VCC).

FIG. 8c shows log-killing trends for microbial biofilms exposed to antimicrobial agents. MBIC is the minimum biofilm inhibitory concentration; MBC_{b} is the minimum bactericidal concentration for biofilms; and MLC_{b} is the minimum lethal concentration for fungal biofilms.

### REFERENCES

1. Wiegand, I., Hilpert, K. & Hancock, R. E. W. Agar and broth dilution methods to determine the minimal inhibitory concentration (MIC) of antimicrobial substances. Nat. Protoc. 3, 163-175 (2008).
2. Karatan, E. & Watnick, P. Signals, regulatory networks, and materials that build and break bacterial biofilms. Microbiol. Mol. Biol. Rev. 73, 310-347 (2009).
3. Stewart, P. S. & Franklin, M. J. Physiological heterogeneity in biofilms. Nat. Rev. Microbiol. 6,199-210 (2008).
4. Boles, B. B., Theondel, M. & Singh, P. K. Self-generated diversity produces ‘insurance effects’ in biofilm communities. Proc. Natl. Acad. Sci. USA. 101, 16630-16635 (2004).
5. Lewis, K. Persister cells, dormancy and infectious disease. Nat. Rev. Microbiol. 5, 48-56 (2007).
6. Stewart, P. S. & Costerton, J. W. The antibiotic resistance of bacteria in biofilms. Lancet 358, 135-138 (2001).
7. Harrison, J. J., Ceri, H. & Turner, R. J. Multimetal resistance and tolerance in microbial biofilms. Nat. Rev. Microbiol. 5, 928-938 (2007).
8. Drenkard, E. & Ausubel, F. M. Pseudomonas biofilm formation and antibioitic resistance are linked to phenotypic variation. Nature 416, 740-743 (2002).
9. Costerton, J. W., Stewart, P. S. & Greenberg, E. P. Bacterial biofilms: a common cause of persistent infections. Science 284, 1318-1322 (1999).
10. Parsek, M. R. & Singh, P. K. Bacterial biofilms: an emerging link to disease pathogenesis. Annu. Rev. Microbiol. 57, 677-701 (2003).
11. Hall-Stoodley, L. et al. Direct detection of bacterial biofilms on the middle-ear mucosa of children with chronic otitis media. J. Am. Med. Assoc. 296, 202-211 (2006).
12. Mandel, E. M. & Casselbrant, M. L. Antibiotics for otitis media with effusion. Minerva Pediatr. 56, 481-495 (2004).
13. Slinger, R. et al. Multiple combination antibiotic susceptibility testing of nontypeable Haemophilus influenzae biofilms. Diagn. Microbiol. Infect. Dis. 56, 247-253 (2006).
14. Pierce, C. G. et al. A simple and reproducible 96-well plate-based method for the formation of fungal biofilms and its application to antifungal susceptibility testing. Nat. Protoc. 3, 1494-1500 (2008).
15. O'Toole, G. A. & Kolter, R. Initiation of biofilm formation in Pseudomonas fluorescens WCS365 proceeds via multiple, convergent signalling pathways: a genetic analysis. Mol. Microbiol. 28, 449-461 (1998).
16. O'Toole, G. A. & Kolter, R. Flagellar and twitching motility are necessary for Pseudomonas aeruginosa biofilm development. Mol. Microbiol. 30, 295-304 (1998).
17. Ceri, H. et al. The Calgary Biofilm Device: new technology for rapid determination of antibiotic susceptibilities in bacterial biofilms. J. Clin. Microbiol. 37, 1771-1776 (1999).
18. Goeres, D. M. et al. Statistical assessment of a laboratory method for growing biofilms. Microbiology 151, 757-762 (2005).
19. Harrison, J. J. et al. Copper and quaternary ammonium cations exert synergistic bactericidal and anti-biofilm activity against Pseudomonas aeruginosa. Antimicrob. Agents Chemother. 52, 2870-2881 (2008).
20. Harrison, J. J. et al. Metal resistance in Candida biofilms. FEMS Microbiol. Ecol. 55, 479-491 (2006).
21. Harrison, J. J., Turner, R. J. & Ceri, H. A subpopulation of Candida albicans and Candida tropicalis biofilm cells are highly tolerant to chelating agents. FEMS Microbiol. Lett. 272, 172-181 (2007).
22. Periasamy, S., Chalmers, N. I., Du-Thumm, L. & Kolenbrander, P. E. Fusobacterium nucleatum ATCC 10953 requires Actinomyces naeslundii ATCC 43146 for growth on saliva in a three-species community that includes Streptococcus oxalis 34. Appl. Environ. Microbiol. 75, 3250-3257 (2009).
23. Periasamy, S. & Kolenbrander, P. E. Aggregatibacter actinomycetemcomitans builds mutualistic biofilm communities with Fuseobacterium nucleatum and Veillonella species in saliva. Infect. Immun. 77, 3542-3551 (2009).
24. Cunliffe, M. & Kertesz, M. A. Autecological properties of soil sphingomonads involved in the degradation of polycyclic aromatic hydrocarbons. Appl. Microbiol. Biotechnol. 72, 1083-1089 (2006).
25. Harrison, J. J. et al. The use of microscopy and three-dimensional visualization to evaluate the structure of microbial biofilms cultivated in the Calgary Biofilm Device. Biol. Proced. Online 8, 194-215 (2006).
26. Gualdi, L. et al. Cellulose modulates biofilm formation by counteracting curli-mediated colonization of solid surfaces in Escherichia coli. Microbiology 154, 2017-2024 (2008).
27. Spoering, A. & Lewis, K. Biofilm and planktonic cells of Pseudomonas aeruginosa have similar resistance to killing by antimicrobials. J. Bacteriol. 183, 6746-6751 (2001).
28. Vanderlinde, E. M. et al. Rhizobium leguminosarum biovar viciae 3841, deficient in 27-hydroxyoctacosanoate-modified lipopolysaccharide, is impaired in desiccation tolerance, biofilm formation and motility. Microbiology 155, 2055-3069 (2009).
29. Goeres, D. M. et al. A method for growing a biofilm under low shear at the air-liquid interface using the drip flow biofilm reactor. Nat. Protoc. 4, 783-788 (2009).
30. Belley, A. et al. Oritavancin kills stationary-phase and biofilm Staphylococcus aureus in vitro. Antimicrob. Agents Chemother. 53, 918-925 (2009).
31. Toutain-Kidd, C. M. et al. Polysorbate 80 inhibition of Pseudomonas aeruginsa biofilm formation and its cleavage by the secreted lipase LipA. Antimicrob. Agents Chemother. 53, 136-145 (2009).
32. Ali, L., Khambaty, F. & Diachenko, G. Investigating the suitability of the Calgary Biofilm Device for assessing the antimicrobial efficacy of new agents. Bioresour. Technol. 97, 1887-1893 (2006).
33. Russel, A. D., Ahonkhai, I. & Rogers, D. T. Microbiological applications of the inactivation of antibiotics and other antimicrobial agents. J. Appl. Bacteriol. 46, 207-245 (1979).
34. Buckingham-Meyer, K., Goeres, D. M. & Hamilton, M. A. Comparative evaluation of biofilm disinfectant efficacy tests. J. Microbiol. Methods 70, 236-244 (2007).
35. ASTM International. E-1054-02: Standard Test Method for Evaluation of Inactivators of Antimicrobial Agents in Annual Book of ASTM Standards Vol. 11.05 (ASTM International, West Conshohocken, Pa., 2004).
36. Harrison, J. J. et al. Chromosomal antioxidant genes have metal ion-specifc roles as determinants of bacterial metal tolerance. Environ. Microbiol. 11, 2491-2509 (2009).
37. Fothergill, A. W. & McGough, D. A. In vitro antifungal susceptibility testing of yeasts. in Clinical Microbiology Procedures Handbook Vol. 1 (eds. Isenberg, H. D. & Hindler, J.) 5.15.11-15.15.16 (ASM Press, Washington, 1995).
38. Garcia-Castillo, M. et al. Differences in biofilm development and antibiotic susceptibility among Streptococcus pneumoniae isolates from cystic fibrosis samples and blood cultures. J. Antimicrob. Chemother. 59, 301-304 (2007).
39. Peeters, E., Nelis, H. J. & Coenye, T. In vitro activity of ceftazidime, ciprofloxacin, meropenem, minocycline, tobramycin and trimethoprim/sulfamethoxazole against planktonic and sessile Burkholderia cepacia complex bacteria. J. Antimicrob. Chemother. 64, 801-809 (2009).
40. Wei, G.-X., Campagna, A. N. & Bobek, L. A. Effect of MUC7 peptides on the growth of bacteria and on Streptococcus mutans biofilm. J. Antimicrob. Chemother. 57, 1100-1109 (2009).
41. De Keersmaecker, S. C. J. et al. Chemical synthesis of (S)-4,5-dihydroxy-2,3-pentanedione, a bacterial signal molecule precursor, and validation of its activity in Salmonella typhimurium. J. Biol. Chem. 280, 19563-19568 (2005).
42. Harrison, J. J. et al. Metal ions may supress or enhance cellular differentiation in Candida albicans and Candida tropicalis biofilms. Appl. Environ. Microbiol. 73, 4940-4949 (2007).
43. Parahitiyawa, N. B. et al. Interspecies variation in Candida biofilm formation studied using the Calgary biofilm device. APMIS 114, 298-306 (2006).
44. Moskowitz, S. M., Foster, J. M., Emerson, J. & Burns, J. L. Clinically feasible biofilm susceptibility assay for isolates of Pseudomonas aeruginosa from patients with cystic fibrosis. J. Clin. Microbiol. 42, 1915-1922 (2004).
45. Harrison, J. J. et al. The chromosomal toxin gene yafQ is a determinant of multidrug tolerance for Escherichia coli growing in a biofilm. Antimicrob. Agents Chemother. 53, 2253-2258 (2009).
46. Brooun, A., Liu, S. & Lewis, K. A dose-response study of antibiotic resistance in Pseudomonas aeruginosa biofilms. Antimicrob. Agents Chemother. 44, 640-646 (2000).
47. Harrison, J. J. et al. Persister cells mediate tolerance to metal oxyanions in Escherichia coli. Microbiology 151, 3181-3195 (2005).
48. Harrison, J. J., Turner, R. J. & Ceri, H. Persister cells, the biofilm matrix and tolerance to metal cations in biofilm and planktonic Pseudomonas aeruginosa. Environ. Microbiol. 7, 981-994 (2005).
49. Harrison, J. J., Turner, R. J. & Ceri, H. High-throughput metal susceptibility testing of microbial biofilms. BMC Microbiol. 5, 53 (2005).
50. Teitzel, G. M. & Parsek, M. R. Heavy metal resistance of biofilm and planktonic Pseudomonas aeruginosa. Appl. Environ. Microbiol. 69, 2313-2320 (2003).
51. O'Neil, M. J., Heckelman, P. E., Koch, C. B. & Roman, K. J. eds The Merck Index: An Encyclopedia of Chemicals, Drugs and Biologicals 14 ed. (Merck Research Laboratories, Whitehouse Station, N.J., USA, 2006).
52. Andrews, J. M. Determination of minimum inhibitory concentrations. J. Antimicrob. Chemother. 48(Suppl. 1): 5-16 (2001).
53. McFarland, J. An instrument for estimating the number of bacteria in suspensions used for calculating the opsonic index and for vaccines. J. Am. Med. Assoc. 14, 1176-1178 (1907).
54. Periasamy, S. & Kolenbrander, P. E. Mutualistic biofilm communities develop with Porphyromonas gingivalis and initial, early and late colonizers of enamel. J. Bacteriol. 191, 6804-6811 (2009).
55. Tomlin, K. L., Coll, O. P. & Ceri, H. Interspecies biofilms of Pseudomonas aeruginosa and Burkholderia cepacia. Can. J. Microbiol. 47, 949-954 (2001).

### Supplemental References for Example 28

1. Periasamy, S., Chalmers, N. I., Du-Thumm, L., and Kolenbrander, P. E. Fusobacterium nucleatum ATCC 10953 requires Actinomyces naeslundii ATCC 43146 for growth on saliva in a three-species community that includes Streptococcus oxalis 34. Appl. Environ. Microbiol. 75, 3250-3257 (2009).
2. Periasamy, S. and Kolenbrander, P. E. Aggregatibacter actinomycetemcomitans builds mutualistic biofilm communities with Fuseobacterium nucleatum and IYeillonella species in saliva. Infect. Immun. 77, 3542-3551 (2009).
3. Olson, M. E. et al. Biofilm bacteria: formation and comparative susceptibility to antibiotics. Can. J. Yet. Res. 66, 86-92 (2002).
4. Carson, L. et al. Antibiofilm activities of 1-alkyl-3-methylimidazolium chloride ionic liquids. Green Chemistry 11, 492-497 (2009).
5. Harrison, J. J. et al. The use of microscopy and three-dimensional visualization to evaluate the structure of microbial biofilms cultivated in the Calgary Biofilm Device. Biol. Proced. Online 8, 194-215 (2006).
6. Harrison, J. J., Turner, R. J., and Ceri, H. A subpopulation of Candida albicans and Candida tropicalis biofilm cells are highly tolerant to chelating agents. FEMSMicrobiol. Lett. 272, 172-181 (2007).
7. Alfa, M. J. and Howie, R. Modeling microbial survival in buildup biofilm for complex medical devices. BMC Infect. Dis. 9, 56 (2009).
8. Parahitiyawa, N. B. et al. Interspecies variation in Candida biofilm formation studied using the Calgary biofilm device. APMIS 114, 298-306 (2006).
9. Harrison, J. J. et al. Metal ions may supress or enhance cellular differentiation in Candida albicans and Candida tropicalis biofilms. Appl. Environ. Microbiol. 73, 4940-4949 (2007).
10. Sandoe, J. A. T. et al. Measurement of ampicillin, vancomycin, linezolid and gentamicin activity against enterococcal biofilms. J. Antimicrob. Chemother. 57, 767-770 (2006).
11. Ceri, H. et al. The Calgary Biofilm Device: New technology for rapid determination of antibiotic susceptibilities in bacterial biofilms. J. Clin. Microbiol. 37, 1771-1776 (1999).
12. Harrison, J. J. et al. Persister cells mediate tolerance to metal oxyanions in Escherichia coli. Microbiology 151, 3181-3195 (2005).
13. Harrison, J. J., Turner, R. J., and Ceri, H. High-throughput metal susceptibility testing of microbial biofilms. BMC Microbiol. 5, 53 (2005).
14. Harrison, J. J. et al. The chromosomal toxin gene yafQ is a determinant of multidrug tolerance for Escherichia coli growing in a biofilm. Antimicrob. Agents Chemother. 53, 2253-2258 (2009).
15. Harrison, J. J. et al. Effects of the twin-arginine translocase on the structure and antimicrobial susceptibility of Escherichia coli biofilms. Can. J. Microbiol. 51, 671-683 (2005).
16. Slinger, R. et al. Multiple combination antibiotic susceptibility testing of nontypeable Haemophilus influenzae biofilms Diagn. Microbiol. Infect. Dis. 56, 247-253 (2006).
17. Mampel, J. et al. Planktonic replication is essential for biofilm formation by Legionella pneumophila in a complex medium under static and dynamic flow conditions Appl. Environ. Microbiol. 72, 2885-2895 (2006).
18. Ali, L., Khambaty, F., and Diachenko, G. Investigating the suitability of the Calgary Biofilm Device for assessing the antimicrobial efficacy of new agents. Bioresource Technology 97, 1887-1893 (2006).
19. Greendyke, R. and Byrd, T. F. Differential antibiotic susceptibility of Mycobacterium abscessus variants in biofilms and macrophages compared to that of planktonic bacteria. Antimicrob. Agents Chemother. 52, 2019-2026 (2008).
20. Bardouniotis, E., Ceri, H., and Olson, M. E. Biofilm formation and biocide susceptibility testing of Mycobacterium fortuitum and Mycobacterium marium. Curr. Microbiol. 46,28-32 (2003).
21. Bardouniotis, E., Huddleston, W., Ceri, H., and Olson, M. E. Characterization of biofilm growth and biocide susceptibility testing of Mycobacterium phlei using the MBECTM assay system FEMS Microbiol. Lett. 203, 263-267 (2001).
22. Periasamy, S. and Kolenbrander, P. E. Mutualistic biofilm communities develop with Porphyromonas gingivalis and initial, early and late colonizers of enamel. J. Bacteriol. 191, 6804-6811 (2009).
23. Jones, S. M., Dang, T. T., and Martinuzzi, R. Use of quorum sensing antagonists to deter the formation of crystalline Proteus mirabilis biofilms. Int. J. Antimicrob. Agents 34, 360-364 (2009).
24. Harrison, J. J. et al. Copper and quaternary ammonium cations exert synergistic bactericidal and anti-biofilm activity against Pseudomonas aeruginosa. Antimicrob. Agents Chemother. 52, 2870-2881 (2008).
25. Harrison, J. J., Ceri, H., Stremick, C., and Turner, R. J. Biofilm susceptibility to metal toxicity. Environ. Microbiol. 6, 1220-1227 (2004).
26. Harrison, J. J., Turner, R. J., and Ceri, H. Persister cells, the biofilm matrix and tolerance to metal cations in biofilm and planktonic Pseudomonas aeruginosa. Environ. Microbiol. 7, 981-994 (2005).
27. Davies, J. A. et al. The GacS sensor kinase controls phenotypic reversion of small colony variants isolated from biofilms of Pseudomonas aeruginosa PA14. FEMS Microbiol. Ecol. 59, 32-46 (2007).
28. Spoering, A. and Lewis, K. Biofilm and planktonic cells of Pseudomonas aeruginosa have similar resistance to killing by antimicrobials. J. Bacteriol. 183, 6746-6751 (2001).
29. Brooun, A., Liu, S., and Lewis, K. A dose-response study of antibiotic resistance in Pseudomonas aeruginosa biofilms. Antimicrob. Agents Chemother. 44, 640-646 (2000).
30. Workentine, M. L. et al. Pseudomonas fluorescens' view of the periodic table. Environ. Microbiol. 10, 238-250 (2007).
31. Lagacb, L., Jacques, M., Mafu, A. A., and Roy, D. Biofilm formation and biocides sensitivity of Pseudomonas marginalis isolated from a maple sap collection system. J. Food Prot. 69, 2411-2416 (2006).
32. Tremaroli, V. et al. Pseudomonas pseudoalcaligenes KF707 upon biofilm formation on a polystyrene surface acquires a strong antibiotic resistance with minor changes in their tolerance to metal cations and metalloid oxyanions Arch. Microbiol. 190, 29-39 (2008).
33. Vanderlinde, E. M. et al. Rhizobium leguminosarum biovar viciae 3841, deficient in 27-hydroxyoctacosanoate-modified lipopolysaccharide, is impaired in desiccation tolerance, biofilm formation and motility. Microbiology 155, 2055-3069 (2009).
34. Vanderlinde, E. M. et al. Identification of a novel ABC transporter required for desiccation tolerance, and biofilm formation in Rhizobium leguminosarum by. viciae 3841. FEMS Microbiol. Ecol. 71, 327-340 (2010).
35. De Keersmaecker, S. C. J. et al. Chemical synthesis of (S)-4,5-dihydroxy-2,3-pentanedione, a bacterial signal molecule precursor, and validation of its activity in Salmonella typhimurium. J. Biol. Chem. 280, 19563-19568 (2005).
36. Cunliffe, M. and Kertesz, M. A. Autecological properties of soil sphingomonads involved in the degradation of polycyclic aromatic hydrocarbons. Appl. Microbiol. Biotechnol. 72, 1083-1089 (2006).
37. Belley, A. et al. Oritavancin kills stationary-phase and biofilm Staphylococcus aureus in vitro. Antimicrob. Agents Chemother. 53, 918-925 (2009).
38. Frank, K. L., Reichert, E. J., Piper, K. E., and Patel, R. In vitro effects of antimicrobial agents on planktonic and biofilm forms of Staphylococcus lugdunensis clinical isolates. Antimicrob. Agents Chemother. 51, 888-895 (2007).
39. Chalmers, N. I., Palmer, R. J. J., Cisar, J. O., and Kolenbrander, P. E. Characterization of a Streptococcus sp.-Veillonella sp. community micromanipulated from dental plaque. J. Bacteriol. 190, 8145-8154 (2008).
40. Shimazu, K. et al. Identification of the Streptococcus gordonii glmM gene encoding phosphoglucosamine mutase and its role in bacterial cell morphology, biofilm formation, and sensitivity to antibiotics. FEMSImmunol. Med. Microbiol. 53, 166-177 (2008).
41. Wei, G.-X., Campagna, A. N., and Bobek, L. A. Effect of MUC7 peptides on the growth of bacteria and on Streptococcus mutans biofilm. J. Antimicrob. Chemother. 57,1100-1109 (2009).

Although a few preferred embodiments have been described, it will be appreciated by those skilled in the art that various changes and modifications might be made without departing from the scope of the invention. The terms and expressions in the preceding specification have been used therein as terms of description and not of limitation, and there is no intention in the use of such terms and expressions of excluding equivalents of the features shown and described or portions thereof, it being recognized as the scope of the invention as defined and limited only by the claims that follow.

1. A method of of developing a diagnostic or susceptibility selection protocol for treating a microbial disease, said protocol being specific for a particular subject, comprising
A. obtaining a sample from a subject;
B. growing a microorganism in the sample in a biofilm formation device to form an optimized biofilm sample;
C. processing the optimized biofilm sample in a biofilm susceptibility device, whereby one or more optimized biofilm samples are exposed to one or more anti-microbial active agents;
D. analyzing the exposed biofilm samples by determining the Minimum Inhibitory Concentration (MIC), Minimum Biofilm Eradication Concentration (MBEC), and Minimum Biocidal Concentration (MBC) values for a microorganism contained in the sample; and
E. selecting one or more active agents effective against the biofilm based on the MIC, MBEC, and MBC values, said one or more active agents being specific for the particular subject.

2. The method of claim 1 wherein establishing optimum growing conditions comprises tailoring growing conditions for at least one specific microorganism, said conditions comprising one or more of the group comprising the surface composition of a substrate, promoting cell adherence to the substrate, the rate of rocking or orbital motion, temperature, cultivation time, inoculum size, atmospheric gases, growth medium, pre-exposure control measurements, reducing contamination, and assessing the biofilm growth for an asymmetric growth pattern.

3. The method of claim 1 wherein establishing optimum conditions for susceptibility testing comprises tailoring susceptibility testing conditions for at least one specific microorganism, said conditions comprising one or more of the group comprising mean cell count, exposure time, recovery medium, reproducible cell density, rinsing medium, optimizing sonication time, and optimizing sonication conditions.

4. The method of claim 1 further comprising calibrating a reactor assembly or parts thereof prior to establishing optimum growing conditions.

5. The method of claim 4 wherein the biofilm is formed from bacteria, fungi, algae, viruses, or parasites; a biofilm from a microorganism that is incorporated within a biofilm as it is formed; a mixed biofilms thereof.

6. The method of claim 5 wherein the microorganism is selected from the group consisting of E. coli, Burkholderia spp., Clostridium spp., Fusobacterium Acinetobacter spp, Proteus spp., Salmonella spp., Stenotrophomonas spp., Pseudomonas spp., Vibrio spp., Yersinia spp., Campylobacter spp., and mixtures or combinations thereof.

7. The method of claim 1 wherein growing a microorganism includes providing a biofilm adherent site having a biofilm growth facilitator.

8. The method of claim 7 wherein the biofilm growth facilitator is selected from the group consisting of host material, poly-l-lysine, hydroxyapatite, collagen, fibronectin, platinum, and combinations thereof.

9. The method of claim 1 wherein exposing the biofilm to one or more active agents comprises exposing the biofilm to a panel of active agents particularly chosen for their possible activity against the microorganism, wherein the active agent is a single active agent or in combination with one of more additional active agents.

10. The method of claim 9 wherein the concentration of active agent tested is a serum MIC breakpoint level.

11. The method of claim 9 wherein the panel is selected from the group consisting of antimicrobial agents against at least one gram negative bacterium and combinations or mixtures thereof; antimicrobial agents against at least one gram positive bacterium and combinations or mixtures thereof; combinations or mixtures of antimicrobial agents against gram positive bacteria and gram negative bacteria; and antimicrobial agents against at least one fungus.

12. The method of claim 1 further comprising using the MBEC, MIC, and MBC values to identify in the microorganism genetic shift , antibiotic resistance, genetic variations, or combinations thereof.

13. (canceled)

14. The method of claim 1 wherein the one or more active agents are frozen, lyophilized, freeze-dried, or vacuum-dried.

15. The method of claim 1 wherein the biofilm susceptibility device comprises a panel or library of active agents, each selected for known effectiveness against a specific microorganism.

16. The method of claim 1 further comprising administering said one or more active agents to the subject having a biofilm-mediated disease or condition.

17. The method of claim 1 further comprising in step D, determining the MIC, MBIC, MBEC, MBCₚ, MBC_{b}, MLCₚ, MLCₚ values, or combinations thereof.

18. The method of claim 1 wherein the microbial disease is a human or animal disease.

19. A treatment regimen for treating an infection mediated by at least one biofilm comprising determining the MIC, MBIC, MBEC, MBCₚ, MBC_{b}, MLCₚ, and MLCₚ values of a biofilm grown from a subject sample, and selecting the active agent or combination of active agents that provide the optimum treatment regimen.
